SEQUENCE LISTING (23 sequences)

SEQ ID NO: 1 - protein, 259 aa, Artificial
  12a2h1-3a-12a2h1 (ALX-0081)
EVQLVESGGGLVQPGGSLRLSCAASGRTFSYNPMGWFRQAPGKGRELVAAISRTGGSTYYPDSVEGRFTISRDNAKRMVYLQMNSLRAEDTAVYYCAAAGVRAEDGRVRTLPSEYTFWGQGTQVTVSSAAAEVQLVESGGGLVQPGGSLRLSCAASGRTFSYNPMGWFRQAPGKGRELVAAISRTGGSTYYPDSVEGRFTISRDNAKRMVYLQMNSLRAEDTAVYYCAAAGVRAEDGRVRTLPSEYTFWGQGTQVTVSS

SEQ ID NO: 2 - protein, 259 aa, Artificial
  12A2-3a-12A2
QVKLEESGGGLVQAGGALRLSCAASGRTFSYNPMGWFRQAPGKERDLVAAISRTGGSTYYPDSVEGRFTISRDNAKRMVYLQMNNLKPEDTAVYYCAAAGVRAEDGRVRTLPSEYTFWGQGTQVTVSSAAAEVQLVESGGGLVQAGGALRLSCAASGRTFSYNPMGWFRQAPGKERDLVAAISRTGGSTYYPDSVEGRFTISRDNAKRMVYLQMNNLKPEDTAVYYCAAAGVRAEDGRVRTLPSEYTFWGQGTQVTVSS

SEQ ID NO: 3 - protein, 265 aa, Artificial
  12A2-GS9-12A2
QVKLEESGGGLVQAGGALRLSCAASGRTFSYNPMGWFRQAPGKERDLVAAISRTGGSTYYPDSVEGRFTISRDNAKRMVYLQMNNLKPEDTAVYYCAAAGVRAEDGRVRTLPSEYTFWGQGTQVTVSSGGGGSGGGSEVQLVESGGGLVQAGGALRLSCAASGRTFSYNPMGWFRQAPGKERDLVAAISRTGGSTYYPDSVEGRFTISRDNAKRMVYLQMNNLKPEDTAVYYCAAAGVRAEDGRVRTLPSEYTFWGQGTQVTVSS

SEQ ID NO: 4 - protein, 286 aa, Artificial
  12A2-GS30-12A2
QVKLEESGGGLVQAGGALRLSCAASGRTFSYNPMGWFRQAPGKERDLVAAISRTGGSTYYPDSVEGRFTISRDNAKRMVYLQMNNLKPEDTAVYYCAAAGVRAEDGRVRTLPSEYTFWGQGTQVTVSSGGGGSGGGGSGGGGSGGGGSGGGGSGGGGSEVQLVESGGGLVQAGGALRLSCAASGRTFSYNPMGWFRQAPGKERDLVAAISRTGGSTYYPDSVEGRFTISRDNAKRMVYLQMNNLKPEGTAVYYCAAAGVRAEDGRVRTLPSEYTFWGQGTQVTVSS

SEQ ID NO: 5 - protein, 247 aa, Artificial
  12A5-3a-12A5
AVQLVESGGGLVQPGGSLRLSCLASGRIFSIGAMGMYRQAPGKQRELVATITSGGSTNYADPVKGRFTISRDGPKNTVYLQMNSLKPEDTAVYYCYANLKQGSYGYRFNDYWGQGTQVTVSSAAAEVQLVESGGGLVQPGGSLRLSCLASGRIFSIGAMGMYRQAPGKQRELVATITSGGSTNYADPVKGRFTISRDGPKNTVYLQMNSLKPEDTAVYYCYANLKQGSYGYRFNDYWGQGTQVTVSS

SEQ ID NO: 6 - protein, 253 aa, Artificial
  12A5-GS9-12A5
AVQLVESGGGLVQPGGSLRLSCLASGRIFSIGAMGMYRQAPGKQRELVATITSGGSTNYADPVKGRFTISRDGPKNTVYLQMNSLKPEDTAVYYCYANLKQGSYGYRFNDYWGQGTQVTVSSGGGGSGGGSEVQLVESGGGLVQPGGSLRLSCLASGRIFSIGAMGMYRQAPGKQRELVATITSGGSTNYADPVKGRFTISRDGPKNTVYLQMNSLKPEDTAVYYCYANLKQGSYGYRFNDYWGQGTQVTVSS

SEQ ID NO: 7 - protein, 274 aa, Artificial
  12A5-GS30-12A5
AVQLVESGGGLVQPGGSLRLSCLASGRIFSIGAMGMYRQAPGKQRELVATITSGGSTNYADPVKGRFTISRDGPKNTVYLQMNSLKPEDTAVYYCYANLKQGSYGYRFNDYWGQGTQVTVSSGGGGSGGGGSGGGGSGGGGSGGGGSGGGGSEVQLVESGGGLVQPGGSLRLSCLASGRIFSIGAMGMYRQAPGKQRELVATITSGGSTNYADPVKGRFTISRDGPKNTVYLQMNSLKPEDTAVYYCYANLKQGSYGYRFNDYWGQGTQVTVSS

SEQ ID NO: 8 - protein, 259 aa, Artificial
  12B6-3a-12B6
QVQLVESGGGLVQAGGALRLSCAASGRTFSYNPMGWFRQAPGKERDVVAAISRTGGSTYYARSVEGRFTISRDNAKRMVYLQMNALKPEDTAVYYCAAAGVRAEDGRVRTLPSEYNFWGQGTQVTVSSAAAEVQLVESGGGLVQAGGALRLSCAASGRTFSYNPMGWFRQAPGKERDVVAAISRTGGSTYYARSVEGRFTISRDNAKRMVYLQMNALKPEDTAVYYCAAAGVRAEDGRVRTLPSEYNFWGQGTQVTVSS

SEQ ID NO: 9 - protein, 265 aa, Artificial
  12B6-GS9-12B6
QVQLVESGGGLVQAGGALRLSCAASGRTFSYNPMGWFRQAPGKERDVVAAISRTGGSTYYARSVEGRFTISRDNAKRMVYLQMNALKPEDTAVYYCAAAGVRAEDGRVRTLPSEYNFWGQGTQVTVSSGGGGSGGGSEVQLVESGGGLVQAGGALRLSCAASGRTFSYNPMGWFRQAPGKERDVVAAISRTGGSTYYARSVEGRFTISRDNAKRMVYLQMNALKPEDTAVYYCAAAGVRAEDGRVRTLPSEYNFWGQGTQVTVSS

SEQ ID NO: 10 - protein, 286 aa, Artificial
  12B6-GS30-12B6
QVQLVESGGGLVQAGGALRLSCAASGRTFSYNPMGWFRQAPGKERDVVAAISRTGGSTYYARSVEGRFTISRDNAKRMVYLQMNALKPEDTAVYYCAAAGVRAEDGRVRTLPSEYNFWGQGTQVTVSSGGGGSGGGGSGGGGSGGGGSGGGGSGGGGSEVQLVESGGGLVQAGGALRLSCAASGRTFSYNPMGWFRQAPGKERDVVAAISRTGGSTYYARSVEGRFTISRDNAKRMVYLQMNALKPEDTAVYYCAAAGVRAEDGRVRTLPSEYNFWGQGTQVTVSS

SEQ ID NO: 11 - protein, 259 aa, Artificial
  12A2H4-3a-12A2H4
EVQLVESGGGLVQPGGSLRLSCAASGRTFSYNPMGWFRQAPGKGRELVAAISRTGGSTYYPDSVEGRFTISRDNAKRSVYLQMNSLRAEDTAVYYCAAAGVRAEDGRVRTLPSEYTFWGQGTQVTVSSAAAEVQLVESGGGLVQPGGSLRLSCAASGRTFSYNPMGWFRQAPGKGRELVAAISRTGGSTYYPDSVEGRFTISRDNAKRSVYLQMNSLRAEDTAVYYCAAAGVRAEDGRVRTLPSEYTFWGQGTQVTVSS

SEQ ID NO: 12 - protein, 259 aa, Artificial
  12B6H2-3a-12B6H2
EVQLVESGGGLVQPGGSLRLSCAASGRTFSYNPMGWFRQAPGKGREVVAAISRTGGSTYYARSVEGRFTISRDNAKRMVYLQMNSLRAEDTAVYYCAAAGVRAEDGRVRTLPSEYNFWGQGTQVTVSSAAAEVQLVESGGGLVQPGGSLRLSCAASGRTFSYNPMGWFRQAPGKGREVVAAISRTGGSTYYARSVEGRFTISRDNAKRMVYLQMNSLRAEDTAVYYCAAAGVRAEDGRVRTLPSEYNFWGQGTQVTVSS

SEQ ID NO: 13 - protein, 265 aa, Artificial
  12A2H1-GS9-12A2H1
EVQLVESGGGLVQPGGSLRLSCAASGRTFSYNPMGWFRQAPGKGRELVAAISRTGGSTYYPDSVEGRFTISRDNAKRMVYLQMNSLRAEDTAVYYCAAAGVRAEDGRVRTLPSEYTFWGQGTQVTVSSGGGGSGGGSEVQLVESGGGLVQPGGSLRLSCAASGRTFSYNPMGWFRQAPGKGRELVAAISRTGGSTYYPDSVEGRFTISRDNAKRMVYLQMNSLRAEDTAVYYCAAAGVRAEDGRVRTLPSEYTFWGQGTQVTVSS

SEQ ID NO: 14 - protein, 265 aa, Artificial
  12A2H4-GS9-12A2H4
EVQLVESGGGLVQPGGSLRLSCAASGRTFSYNPMGWFRQAPGKGRELVAAISRTGGSTYYPDSVEGRFTISRDNAKRSVYLQMNSLRAEDTAVYYCAAAGVRAEDGRVRTLPSEYTFWGQGTQVTVSSGGGGSGGGSEVQLVESGGGLVQPGGSLRLSCAASGRTFSYNPMGWFRQAPGKGRELVAAISRTGGSTYYPDSVEGRFTISRDNAKRSVYLQMNSLRAEDTAVYYCAAAGVRAEDGRVRTLPSEYTFWGQGTQVTVSS

SEQ ID NO: 15 - protein, 265 aa, Artificial
  12B6H2-GS9-12B6H2
EVQLVESGGGLVQPGGSLRLSCAASGRTFSYNPMGWFRQAPGKGREVVAAISRTGGSTYYARSVEGRFTISRDNAKRMVYLQMNSLRAEDTAVYYCAAAGVRAEDGRVRTLPSEYNFWGQGTQVTVSSGGGGSGGGSEVQLVESGGGLVQPGGSLRLSCAASGRTFSYNPMGWFRQAPGKGREVVAAISRTGGSTYYARSVEGRFTISRDNAKRMVYLQMNSLRAEDTAVYYCAAAGVRAEDGRVRTLPSEYNFWGQGTQVTVSS

SEQ ID NO: 16 - protein, 286 aa, Artificial
  12A2H1-GS30-12A2H1
EVQLVESGGGLVQPGGSLRLSCAASGRTFSYNPMGWFRQAPGKGRELVAAISRTGGSTYYPDSVEGRFTISRDNAKRMVYLQMNSLRAEDTAVYYCAAAGVRAEDGRVRTLPSEYTFWGQGTQVTVSSGGGGSGGGGSGGGGSGGGGSGGGGSGGGGSEVQLVESGGGLVQPGGSLRLSCAASGRTFSYNPMGWFRQAPGKGRELVAAISRTGGSTYYPDSVEGRFTISRDNAKRMVYLQMNSLRAEDTAVYYCAAAGVRAEDGRVRTLPSEYTFWGQGTQVTVSS

SEQ ID NO: 17 - protein, 286 aa, Artificial
  12A2H4-GS30-12A2H4
EVQLVESGGGLVQPGGSLRLSCAASGRTFSYNPMGWFRQAPGKGRELVAAISRTGGSTYYPDSVEGRFTISRDNAKRSVYLQMNSLRAEDTAVYYCAAAGVRAEDGRVRTLPSEYTFWGQGTQVTVSSGGGGSGGGGSGGGGSGGGGSGGGGSGGGGSEVQLVESGGGLVQPGGSLRLSCAASGRTFSYNPMGWFRQAPGKGRELVAAISRTGGSTYYPDSVEGRFTISRDNAKRSVYLQMNSLRAEDTAVYYCAAAGVRAEDGRVRTLPSEYTFWGQGTQVTVSS

SEQ ID NO: 18 - protein, 286 aa, Artificial
  12B6H2-GS30-12B6H2
EVQLVESGGGLVQPGGSLRLSCAASGRTFSYNPMGWFRQAPGKGREVVAAISRTGGSTYYARSVEGRFTISRDNAKRMVYLQMNSLRAEDTAVYYCAAAGVRAEDGRVRTLPSEYNFWGQGTQVTVSSGGGGSGGGGSGGGGSGGGGSGGGGSGGGGSEVQLVESGGGLVQPGGSLRLSCAASGRTFSYNPMGWFRQAPGKGREVVAAISRTGGSTYYARSVEGRFTISRDNAKRMVYLQMNSLRAEDTAVYYCAAAGVRAEDGRVRTLPSEYNFWGQGTQVTVSS

SEQ ID NO: 19 - protein, 128 aa, Artificial
  12A2h1
EVQLVESGGGLVQPGGSLRLSCAASGRTFSYNPMGWFRQAPGKGRELVAAISRTGGSTYYPDSVEGRFTISRDNAKRMVYLQMNSLRAEDTAVYYCAAAGVRAEDGRVRTLPSEYTFWGQGTQVTVSS

SEQ ID NO: 20 - protein, 128 aa, Artificial
  12A2
QVKLEESGGGLVQAGGALRLSCAASGRTFSYNPMGWFRQAPGKERDLVAAISRTGGSTYYPDSVEGRFTISRDNAKRMVYLQMNNLKPEDTAVYYCAAAGVRAEDGRVRTLPSEYTFWGQGTQVTVSS

SEQ ID NO: 21 - protein, 122 aa, Artificial
  12A5
AVQLVESGGGLVQPGGSLRLSCLASGRIFSIGAMGMYRQAPGKQRELVATITSGGSTNYADPVKGRFTISRDGPKNTVYLQMNSLKPEDTAVYYCYANLKQGSYGYRFNDYWGQGTQVTVSS

SEQ ID NO: 22 - protein, 128 aa, Artificial
  12B6
QVQLVESGGGLVQAGGALRLSCAASGRTFSYNPMGWFRQAPGKERDVVAAISRTGGSTYYARSVEGRFTISRDNAKRMVYLQMNALKPEDTAVYYCAAAGVRAEDGRVRTLPSEYNFWGQGTQVTVSS

SEQ ID NO: 23 - protein, 2800 aa, Homo sapiens
MIPARFAGVLLALALILPGTLCAEGTRGRSSTARCSLFGSDFVNTFDGSMYSFAGYCSYLLAGGCQKRSFSIIGDFQNGKRVSLSVYLGEFFDIHLFVNGTVTQGDQRVSMPYASKGLYLETEAGYYKLSGEAYGFVARIDGSGNFQVLLSDRYFNKTCGLCGNFNIFAEDDFMTQEGTLTSDPYDFANSWALSSGEQWCERASPPSSSCNISSGEMQKGLWEQCQLLKSTSVFARCHPLVDPEPFVALCEKTLCECAGGLECACPALLEYARTCAQEGMVLYGWTDHSACSPVCPAGMEYRQCVSPCARTCQSLHINEMCQERCVDGCSCPEGQLLDEGLCVESTECPCVHSGKRYPPGTSLSRDCNTCICRNSQWICSNEECPGECLVTGQSHFKSFDNRYFTFSGICQYLLARDCQDHSFSIVIETVQCADDRDAVCTRSVTVRLPGLHNSLVKLKHGAGVAMDGQDIQLPLLKGDLRIQHTVTASVRLSYGEDLQMDWDGRGRLLVKLSPVYAGKTCGLCGNYNGNQGDDFLTPSGLAEPRVEDFGNAWKLHGDCQDLQKQHSDPCALNPRMTRFSEEACAVLTSPTFEACHRAVSPLPYLRNCRYDVCSCSDGRECLCGALASYAAACAGRGVRVAWREPGRCELNCPKGQVYLQCGTPCNLTCRSLSYPDEECNEACLEGCFCPPGLYMDERGDCVPKAQCPCYYDGEIFQPEDIFSDHHTMCYCEDGFMHCTMSGVPGSLLPDAVLSSPLSHRSKRSLSCRPPMVKLVCPADNLRAEGLECTKTCQNYDLECMSMGCVSGCLCPPGMVRHENRCVALERCPCFHQGKEYAPGETVKIGCNTCVCRDRKWNCTDHVCDATCSTIGMAHYLTFDGLKYLFPGECQYVLVQDYCGSNPGTFRILVGNKGCSHPSVKCKKRVTILVEGGEIELFDGEVNVKRPMKDETHFEVVESGRYIILLLGKALSVVWDRHLSISVVLKQTYQEKVCGLCGNFDGIQNNDLTSSNLQVEEDPVDFGNSWKVSSQCADTRKVPLDSSPATCHNNIMKQTMVDSSCRILTSDVFQDCNKLVDPEPYLDVCIYDTCSCESIGDCACFCDTIAAYAHVCAQHGKWTWRTATLCPQSCEERNLRENGYECEWRYNSCAPACQVTCQHPEPLACPVQCVEGCHAHCPPGKILDELLQTCVDPEDCPVCEVAGRRFASGKKVTLNPSDPEHCQICHCDVVNLTCEACQEPGGLVVPPTDAPVSPTTLYVEDISEPPLHDFYCSRLLDLVFLLDGSSRLSEAEFEVLKAFVVDMMERLRISQKWVRVAWEYHDGSHAYIGLKDRKRPSELRRIASQVKYAGSQVASTSEVLKYTLFQIFSKIDRPEASRIALLLMASQEPQRMSRNFVRYVQGKKKKVIVIPVGIGPHANLKQIRLIEKQAPENKAFVLSSVDELEQQRDEIVSYLCDLAPEAPPPTLPPHMAQVTVGPGLRNSMVLDVAFVLEGSDKIGEADFNRSKEFMEEVIQRMDVGQDSIHVTVLQYSYMVTVEYPFSEAQSKGDILQRVREIRYQGGNRTNTGLALRYLSDHSFLVSQGDREQAPNLVYMVTGNPASDEIKRLPGDIQWPIGVGPNANVQELERIGWPNAPILIQDFETLPREAPDLVLQRCCSGEGLQIPTLSPAPDCSQPLDVILLLDGSSSFPASYFDEMKSFAKAFISKANIGPRLTQVSVLQYGSITTIDVPWNVVPEKAHLLSLVDVMQREGGPSQIGDALGFAVRYLTSEMHGARPGASKAVVILVTDVSVDSVDAAADAARSNRVTVFPIGIGDRYDAAQLRILAGPAGDSNVVKLQRIEDLPTMVTLGNSFLHKLCSGFVRICMDEDGNEKRPGDVWTLPDQCHTVTCQPDGQTLLKSHRVNCDRGLRPSCPNSQSPVKVEETCGCRWTCPCVCTGSSTRHIVTFDGQNFKLTGSCSYVLFQNKEQDLEVILHNGACSPGARQGCMKSIEVKHSALSVELHSDMEVTVNGRLVSVPYVGGNMEVNVYGAIMHEVRFNHLGHIFTFTPQNNEFQLQLSPKTFASKTYGLCGICDENGANDFMLRDGTVTTDWKTLVQEWTVQRPGQTCQPILEEQCLVPDSSHCQVLLLPLFAECHKVLAPATFYAICQQDSCHQEQVCEVIASYAHLCRTNGVCVDWRTPDFCAMSCPPSLVYNHCEHGCPRHCDGNVSSCGDHPSEGCFCPPDKVMLEGSCVPEEACTQCIGEDGVQHQFLEAWVPDHQPCQICTCLSGRKVNCTTQPCPTAKAPTCGLCEVARLRQNADQCCPEYECVCDPVSCDLPPVPHCERGLQPTLTNPGECRPNFTCACRKEECKRVSPPSCPPHRLPTLRKTQCCDEYECACNCVNSTVSCPLGYLASTATNDCGCTTTTCLPDKVCVHRSTIYPVGQFWEEGCDVCTCTDMEDAVMGLRVAQCSQKPCEDSCRSGFTYVLHEGECCGRCLPSACEVVTGSPRGDSQSSWKSVGSQWASPENPCLINECVRVKEEVFIQQRNVSCPQLEVPVCPSGFQLSCKTSACCPSCRCERMEACMLNGTVIGPGKTVMIDVCTTCRCMVQVGVISGFKLECRKTTCNPCPLGYKEENNTGECCGRCLPTACTIQLRGGQIMTLKRDETLQDGCDTHFCKVNERGEYFWEKRVTGCPPFDEHKCLAEGGKIMKIPGTCCDTCEEPECNDITARLQYVKVGSCKSEVEVDIHYCQGKCASKAMYSIDINDVQDQCSCCSPTRTEPMQVALHCTNGSVVYHEVLNAMECKCSPRKCSK

(19) United States
(12) Patent Application Publication
(10) Pub. No.: US 2012/0321640 A1
(43) Pub. Date: Dec. 20, 2012

(54) VON WILLEBRAND FACTOR SPECIFIC BINDING AGENTS AND USES THEREOF

(75) Inventors: Maarten Van Roy, Merelbeke (BE); Hans Ulrichts, Kortrijk (BE)

(73) Assignee: Ablynx N.V., Zwijnaarde (BE)

(21) Appl. No.: 13/512,126

(22) PCT Filed: Nov. 25, 2010
(86) PCT No.: PCT/EP10/68208

§ 371 Date: Sep. 5, 2012

Related U.S. Application Data
(60) Provisional application No. 61/265,508, filed on Dec. 1, 2009.

(51) Int. Cl. A61K 39/395; A61P 11/00; A61P 9/10; C07K 16/36; A61P 7/02

(52) U.S. Cl. 424/158.1; 530/389.3

(57) ABSTRACT

The invention provides new uses, compositions and methods of administration for specific binding agents to von Wiliebrand Factor (vWF) in patients with thromboembolic disorders and in particular new combined uses with thrombolytic agents such as tissue plasminogen activator in patients with thromboembolic disorders such as e.g. ischemic stroke. Furthermore, a new group of vWF binding agents and an improved Middle Cerebral Artery Thrombosis Model in guinea pigs to study the effects of stroke such as ischemia (oxygen and glucose depriviation) and hemorrhage (bleeding), in particular hemorrhage, are provided.

The invention provides new uses, compositions and methods of administration for specific binding agents to von Willebrand Factor (vWF) in patients with thromboembolic disorders and in particular new combined uses with thrombolytic agents such as tissue plasminogen activator in patients with thromboembolic disorders such as e.g. ischemic stroke. Furthermore, a new group of vWF binding agents and an improved Middle Cerebral Artery Thrombosis Model in guinea pigs to study the effects of stroke such as ischemia (oxygen and glucose depriviation) and hemorrhage (bleeding), in particular hemorrhage, are provided.

### BACKGROUND OF THE INVENTION

A stroke is the rapidly developing loss of brain function(s) due to disturbance in the blood supply to the brain. This can be due to ischemia caused by thrombosis or embolism (80% of all reported cases) or due to hemorrhage (20%). Some hemorrhages develop inside areas of ischemia. As results, the affected area of the brain is unable to function, leading to inability to move one or more limbs, inability to understand or formulate speech, or inability to see one side of the visual field. Stroke is the leading cause of adult disability in the US and Europe. It is the second most common cause of death, the first being heart attacks and third being cancer. The only therapy available is recombinant tissue plasminogen activator (herein also referred to as “rt-PA”), but side effects such as e.g. bleeding and limited beneficial time interval limit its use.

### SUMMARY OF THE INVENTION

It has recently been suggested that the GPIb-IX-V-von Willebrand factor (herein also referred to as “vWF”) pathway is critically involved in ischemic stroke (Kleinschnitz et al., 2009, Blood, Vol. 113, pages 3600-3603). Moreover, deficiency or reduction of vWF by the vWF cleaving protease ADAMTS13 reduces ischemic brain injury in experimental stroke (Zhao et al., 2009, Blood, Vol. 114, pages 3329-3334). Furthermore, it has been shown that the anti-platelet drug “ALX-0081” (SEQ ID NO: 1) that is a vWF binding agent comprising two identical Nanobodies directed against vWF, interrupts the binding between vWF and platelets, i.e. interrupts binding between the so called A1 domain of vWF and the platelet glycoprotein Ib-IX-V receptor complex (herein also referred to as “GPIb receptor”) of the platelets, and that application of said vWF binding agent prevents thrombus formation in a baboon FOLTS' model (see e.g. Example 18 of WO2006/122825 A2).

It has now been found surprisingly that the combined use of i) a specific anti-platelet drug, i.e. an anti-platelet vWF binding agent, and ii) a thrombolytic drug synergistically reduces thrombus formation in a thromboembolic disorder such as e.g. a myocardial infarction, ischemic stroke, deep vein thrombosis or pulmonary embolism. Indeed the present invention surprisingly provides that thrombolytic drugs, such as rtPA, when combined with an anti-adhesive agent such as e.g. an anti-vWF agent can be used in a broader dose regimen range (lower dose and/or longer treatment window) for the treatment of thromboembolic disorders than the skilled person in the art would have expected.

For example, ALX-0081 (SEQ ID NO: 1) has been found to significantly reduce the ischemic brain damage while no increased intracerebral bleeding was observed in the photochemically induced endothelial damage of the middle cerebral artery (herein also referred to as “MCA”). In contrast to rtPA monotherapy, ALX-0081 monotherapy or in combination with rtPA was able to induce a complete reperfusion of the MCA after injury in the same model.

Accordingly the present invention provides a method for the treatment of a thromboembolic disorder such as e.g. a myocardial infarction, ischemic stroke, deep vein thrombosis or pulmonary embolism, preferably ischemic stroke such as acute ischemic stroke; in patient(s), preferably human(s), in need thereof, wherein said treatment comprises administering
i) an effective dose regimen of an anti vWF agent, e.g. an A1 vWF binding agent, a vWF binding agent with the epitope of 12a2h1 (SEQ ID NO: 19) or ALX-0081 (SEQ ID NO: 1); and
ii) a low dose regimen of a thrombolytic agent, e.g. such as rtPA, to said patient; and
   wherein optionally the time point when the anti-adhesive agent and thrombolytic agent is administered is later than indicated in the case where an anti-thrombolytic agent is administered alone, e.g. later than the standard of care limit of 3 hours or shorter within the event for a standard of care dose of rt-PA administered intravenously (or e.g. later than the standard of care limit of a 6 h or shorter within the event for a standard of care dose of rt-PA administered on site).

Moreover, the present invention provides a method for the treatment of a thromboembolic disorder such as e.g. a myocardial infarction, ischemic stroke, deep vein thrombosis or pulmonary embolism, preferably ischemic stroke such as acute ischemic stroke; in patient(s), preferably human(s), in need thereof, wherein said treatment comprises the inhibition of reocclusion in said patient(s) treated with a thrombolytic agent, e.g. such as rtPA, by administering to said patient(s) an effective dose regimen of an anti vWF agent, e.g. an A1 vWF binding agent, a vWF binding agent with the epitope of 12a2h1 (SEQ ID NO: 19) or ALX-0081 (SEQ ID NO: 1).

Moreover, the present invention provides a method for the treatment of a thromboembolic disorder such as e.g. a myocardial infarction, ischemic stroke, deep vein thrombosis or pulmonary embolism, preferably ischemic stroke such as acute ischemic stroke; in patient(s), preferably human(s), in need thereof, wherein said patient(s) has rtPA resistant thrombi, and wherein said treatment comprises the administration to said patient(s) of an effective dose regimen of an anti vWF agent, e.g. an A1 vWF binding agent, a vWF binding agent with the epitope of 12a2h1 (SEQ ID NO: 19) or ALX-0081 (SEQ ID NO: 1).

Equivalent uses, combinations and pharmaceutical compositions related to the anti vWF agent and thrombolytic agent as outlined in the method above and herein are also provided.

The invention further provides an anti vWF agent of a particular epitope, wherein the anti vWF agents having identical CDRs from any of the nanobodies 12a2 (SEQ ID NO:20), 12a5 (SEQ ID NO:21), and/or 12b6 (SEQ ID NO:22), are disclaimed; and wherein said binding agent interacts with at least certain specified amino acid residues on the A1 domain of vWF.

The invention yet further provides an in vitro screening method using the epitope information described in this invention.

### BRIEF DESCRIPTION OF THE FIGURES

FIG. 1 shows the profiles of ALX-0081 administration; (A) depicts the PK profiles of ALX-0081 administration, (B) depicts the RICO profile of ALX-0081 administration.

FIG. 2 shows the profiles of damage to the MCA (A) as percentage of cerebral blood flow, (B) as percentage of damaged area.

FIG. 3 shows the cerebral blood flow (CBF) indicated in tissue perfusion units (TPU) for (A) ALX-0081, (B) rtPA, (C) ALX-0081+rtPA.

FIG. 4 shows the analysis of brain damage (A) as percentage of ischemic area, (B) as percentage of increase in brain damage.

FIG. 5 shows the bleeding times (A) at administration, (B) 30 minutes after administration, (C) 120 minutes after administration.

FIG. 6 shows the location of the A1-vWF sequence within in the vWF sequence.

FIG. 7 shows the sequence of the 12a2h1 vWF binder.

FIG. 8 shows the structure of the A1-vWF:12a2h1 complex.

### DETAILED DESCRIPTION OF THE INVENTION

### 1. Definitions

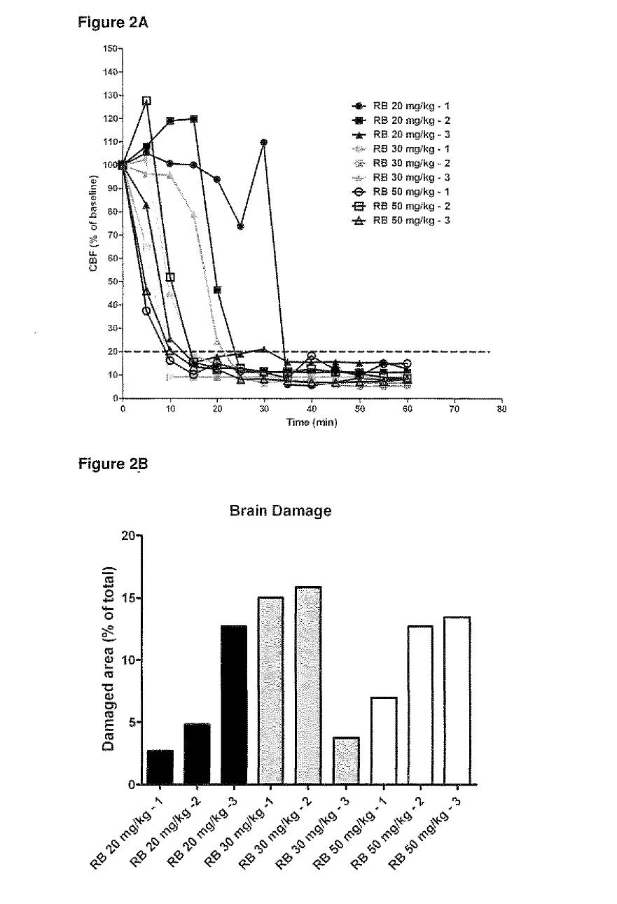

In the present description, examples and claims:
a) Unless indicated or defined otherwise, all terms used have their usual meaning in the art, which will be clear to the skilled person. Reference is for example made to the standard handbooks mentioned in paragraph a) on page 46 of WO 08/020,079.
b) Unless indicated otherwise, the terms “immunoglobulin sequence”, “sequence”, “nucleotide sequence” and “nucleic acid” are as described in paragraph b) on page 46 of WO 08/020,079.
c) Unless indicated otherwise, all methods, steps, techniques and manipulations that are not specifically described in detail can be performed and have been performed in a manner known per se, as will be clear to the skilled person. Reference is for example again made to the standard handbooks and the general background art mentioned herein and to the further references cited therein; as well as to for example the following reviews Presta, Adv. Drug Deliv. Rev. 2006, 58 (5-6): 640-56; Levin and Weiss, Mol. Biosyst. 2006, 2(1): 49-57; Irving et al., J. Immunol. Methods, 2001, 248(1-2), 31-45; Schmitz et al., Placenta, 2000, 21 Suppl. A, S106-12, Gonzales et al., Tumour Biol., 2005, 26(1), 31-43, which describe techniques for protein engineering, such as affinity maturation and other techniques for improving the specificity and other desired properties of proteins such as immunoglobulins.
d) Amino acid residues will be indicated according to the standard three-letter or one-letter amino acid code. Reference is made to Table A-2 on page 48 of the International application WO 08/020,079 of Ablynx N.V. entitled “Amino acid sequences directed against IL-6R and polypeptides comprising the same for the treatment of diseases and disorders associated with IL-6 mediated signalling”.
e) For the purposes of comparing two or more nucleotide sequences, the percentage of “sequence identity” between a first nucleotide sequence and a second nucleotide sequence may be calculated or determined as described in paragraph e) on page 49 of WO 08/020,079 (incorporated herein by reference), such as by dividing [the number of nucleotides in the first nucleotide sequence that are identical to the nucleotides at the corresponding positions in the second nucleotide sequence] by [the total number of nucleotides in the first nucleotide sequence] and multiplying by [100%], in which each deletion, insertion, substitution or addition of a nucleotide in the second nucleotide sequence—compared to the first nucleotide sequence—is considered as a difference at a single nucleotide (position); or using a suitable computer algorithm or technique, again as described in paragraph e) on pages 49 of WO 08/020,079 (incorporated herein by reference).
f) For the purposes of comparing two or more amino acid sequences, the percentage of “sequence identity” between a first amino acid sequence and a second amino acid sequence (also referred to herein as “amino acid identity”) may be calculated or determined as described in paragraph f) on pages 49 and 50 of WO 08/020,079 (incorporated herein by reference), such as by dividing [the number of amino acid residues in the first amino acid sequence that are identical to the amino acid residues at the corresponding positions in the second amino acid sequence] by [the total number of amino acid residues in the first amino acid sequence] and multiplying by [100%], in which each deletion, insertion, substitution or addition of an amino acid residue in the second amino acid sequence—compared to the first amino acid sequence—is considered as a difference at a single amino acid residue (position), i.e. as an “amino acid difference” as defined herein; or using a suitable computer algorithm or technique, again as described in paragraph f) on pages 49 and 50 of WO 08/020,079 (incorporated herein by reference).
   Also, in determining the degree of sequence identity between two amino acid sequences, the skilled person may take into account so-called “conservative” amino acid substitutions, as described on page 50 of WO 08/020,079.
   Any amino acid substitutions applied to the polypeptides described herein may also be based on the analysis of the frequencies of amino acid variations between homologous proteins of different species developed by Schulz et al., Principles of Protein Structure, Springer-Verlag, 1978, on the analyses of structure forming potentials developed by Chou and Fasman, Biochemistry 13: 211, 1974 and Adv. Enzymol., 47: 45-149, 1978, and on the analysis of hydrophobicity patterns in proteins developed by Eisenberg et al., Proc. Nad. Acad. Sci. USA 81: 140-144, 1984; Kyte & Doolittle; J Molec. Biol. 157: 105-132, 198 1, and Goldman et al., Ann. Rev. Biophys. Chem. 15: 321-353, 1986, all incorporated herein in their entirety by reference. Information on the primary, secondary and tertiary structure of Nanobodies is given in the description herein and in the general background art cited above. Also, for this purpose, the crystal structure of a V_{HH} domain from a llama is for example given by Desmyter et al., Nature Structural Biology, Vol. 3, 9, 803 (1996); Spinelli et al., Natural Structural Biology (1996); 3, 752-757; and Decanniere et al., Structure, Vol. 7, 4, 361 (1999). Further information about some of the amino acid residues that in conventional V_{H} domains form the V_{H}/V_{L} interface and potential camelizing substitutions on these positions can be found in the prior art cited above.
g) Amino acid sequences and nucleic acid sequences are said to be “exactly the same” if they have 100% sequence identity (as defined herein) over their entire length.
h) When comparing two amino acid sequences, the term “amino acid difference” refers to an insertion, deletion or substitution of a single amino acid residue on a position of the first sequence, compared to the second sequence; it being understood that two amino acid sequences can contain one, two or more such amino acid differences.
i) When a nucleotide sequence or amino acid sequence is said to “comprise” another nucleotide sequence or amino acid sequence, respectively, or to “essentially consist of” another nucleotide sequence or amino acid sequence, this has the meaning given in paragraph i) on pages 51-52 of WO 08/020,079.
j) The term “in essentially isolated form” has the meaning given to it in paragraph j) on pages 52 and 53 of WO 08/020,079.
k) The terms “domain” and “binding domain” have the meanings given to it in paragraph k) on page 53 of WO 08/020,079.
l) The terms “antigenic determinant” and “epitope”, which may also be used interchangeably herein, have the meanings given to it in paragraph l) on page 53 of WO 08/020,079.
m) As further described in paragraph m) on page 53 of WO 08/020,079, an amino acid sequence (such as a Nanobody, an antibody, a polypeptide of the invention, or generally an antigen binding protein or polypeptide or a fragment thereof) that can (specifically) bind to, that has affinity for and/or that has specificity for a specific antigenic determinant, epitope, antigen or protein (or for at least one part, fragment or epitope thereof) is said to be “against” or “directed against” said antigenic determinant, epitope, antigen or protein.
n) The term “specificity” has the meaning given to it in paragraph n) on pages 53-56 of WO 08/020,079; and as mentioned therein refers to the number of different types of antigens or antigenic determinants to which a particular antigen-binding molecule or antigen-binding protein (such as a Nanobody or a polypeptide of the invention) molecule can bind. The specificity of an antigen-binding protein can be determined based on affinity and/or avidity, as described on pages 53-56 of WO 08/020,079 (incorporated herein by reference), which also describes some preferred techniques for measuring binding between an antigen-binding molecule (such as a Nanobody or polypeptide of the invention) and the pertinent antigen. Typically, antigen-binding proteins (such as the amino acid sequences, Nanobodies and/or polypeptides of the invention) will bind to their antigen with a dissociation constant (K_{D}) of 10⁻⁵ to 10⁻¹² moles/liter or less, and preferably 10⁻⁷ to 10⁻¹² moles/liter or less and more preferably 10⁻⁸ to 10⁻¹² moles/liter (i.e. with an association constant (K_{A}) of 10⁵ to 10¹² liter/moles or more, and preferably 10⁷ to 10¹² liter/moles or more and more preferably 10⁸ to 10¹² liter/moles). Any K_{D} value greater than 10⁴ mol/liter (or any K_{A} value lower than 10⁴ M⁻¹) liters/mol is generally considered to indicate non-specific binding. Preferably, a monovalent immunoglobulin sequence of the invention will bind to the desired antigen with an affinity less than 500 nM, preferably less than 200 nM, more preferably less than 10 nM, such as less than 500 μM. Specific binding of an antigen-binding protein to an antigen or antigenic determinant can be determined in any suitable manner known per se, including, for example, Scatchard analysis and/or competitive binding assays, such as radioimmunoassays (RIA), enzyme immunoassays (EIA) and sandwich competition assays, and the different variants thereof known per se in the art; as well as the other techniques mentioned herein. As will be clear to the skilled person, and as described on pages 53-56 of WO 08/020,079, the dissociation constant may be the actual or apparent dissociation constant. Methods for determining the dissociation constant will be clear to the skilled person, and for example include the techniques mentioned on pages 53-56 of WO 08/020,079.
o) The half-life of an amino acid sequence, compound or polypeptide of the invention can generally be defined as described in paragraph o) on page 57 of WO 08/020,079 and as mentioned therein refers to the time taken for the serum concentration of the amino acid sequence, compound or polypeptide to be reduced by 50%, in vivo, for example due to degradation of the sequence or compound and/or clearance or sequestration of the sequence or compound by natural mechanisms. The in vivo half-life of an amino acid sequence, compound or polypeptide of the invention can be determined in any manner known per se, such as by pharmacokinetic analysis. Suitable techniques will be clear to the person skilled in the art, and may for example generally be as described in paragraph o) on page 57 of WO 08/020,079. As also mentioned in paragraph o) on page 57 of WO 08/020,079, the half-life can be expressed using parameters such as the t1/2-alpha, t1/2-beta and the area under the curve (AUC). Reference is for example made to the Experimental Part below, as well as to the standard handbooks, such as Kenneth, A et al: Chemical Stability of Pharmaceuticals: A Handbook for Pharmacists and Peters et al, Pharmacokinete analysis: A Practical Approach (1996). Reference is also made to “Pharmacokinetics”, M Gibaldi & D Perron, published by Marcel Dekker, 2nd Rev. edition (1982). The terms “increase in half-life” or “increased half-life” as also as defined in paragraph o) on page 57 of WO 08/020,079 and in particular refer to an increase in the t1/2-beta, either with or without an increase in the t1/2-alpha and/or the AUC or both.
p) in the context of the present invention, “modulating” or “to modulate” generally means either reducing or inhibiting the activity of, or alternatively increasing the activity of, a target or antigen, as measured using a suitable in vitro, cellular or in vivo assay. In particular, “modulating” or “to modulate” may mean either reducing or inhibiting the activity of, or alternatively increasing a (relevant or intended) biological activity of, a target or antigen, as measured using a suitable in vitro, cellular or in vivo assay (which will usually depend on the target or antigen involved), by at least 1%, preferably at least 5%, such as at least 10% or at least 25%, for example by at least 50%, at least 60%, at least 70%, at least 80%, or 90% or more, compared to activity of the target or antigen in the same assay under the same conditions but without the presence of the construct of the invention.
   As will be clear to the skilled person, “modulating” may also involve effecting a change (which may either be an increase or a decrease) in affinity, avidity, specificity and/or selectivity of a target or antigen for one or more of its ligands, binding partners, partners for association into a homomultimeric or heteromultimeric form, or substrates; and/or effecting a change (which may either be an increase or a decrease) in the sensitivity of the target or antigen for one or more conditions in the medium or surroundings in which the target or antigen is present (such as pH, ion strength, the presence of co-factors, etc.), compared to the same conditions but without the presence of the construct of the invention. As will be clear to the skilled person, this may again be determined in any suitable manner and/or using any suitable assay known per se, depending on the target or antigen involved.
   “Modulating” may also mean effecting a change (i.e. an activity as an agonist, as an antagonist or as a reverse agonist, respectively, depending on the target or antigen and the desired biological or physiological effect) with respect to one or more biological or physiological mechanisms, effects, responses, functions, pathways or activities in which the target or antigen (or in which its substrate(s), ligand(s) or pathway(s) are involved, such as its signalling pathway or metabolic pathway and their associated biological or physiological effects) is involved. Again, as will be clear to the skilled person, such an action as an agonist or an antagonist may be determined in any suitable manner and/or using any suitable (in vitro and usually cellular or in assay) assay known per se, depending on the target or antigen involved. In particular, an action as an agonist or antagonist may be such that an intended biological or physiological activity is increased or decreased, respectively, by at least 1%, preferably at least 5%, such as at least 10% or at least 25%, for example by at least 50%, at least 60%, at least 70%, at least 80%, or 90% or more, compared to the biological or physiological activity in the same assay under the same conditions but without the presence of the construct of the invention.
   Modulating may for example also involve allosteric modulation of the target or antigen; and/or reducing or inhibiting the binding of the target or antigen to one of its substrates or ligands and/or competing with a natural ligand, substrate for binding to the target or antigen. Modulating may also involve activating the target or antigen or the mechanism or pathway in which it is involved.
   Modulating may for example also involve effecting a change in respect of the folding or confirmation of the target or antigen, or in respect of the ability of the target or antigen to fold, to change its confirmation (for example, upon binding of a ligand), to associate with other (sub)units, or to disassociate. Modulating may for example also involve effecting a change in the ability of the target or antigen to transport other compounds or to serve as a channel for other compounds (such as ions).
   Modulating may be reversible or irreversible, but for pharmaceutical and pharmacological purposes will usually be in a reversible manner.
q) In respect of a target or antigen, the term “interaction site” on the target or antigen means a site, epitope, antigenic determinant, part, domain or stretch of amino acid residues on the target or antigen that is a site for binding to a ligand, receptor or other binding partner, a catalytic site, a cleavage site, a site for allosteric interaction, a site involved in multimerisation (such as homomerization or heterodimerization) of the target or antigen; or any other site, epitope, antigenic determinant, part, domain or stretch of amino acid residues on the target or antigen that is involved in a biological action or mechanism of the target or antigen. More generally, an “interaction site” can be any site, epitope, antigenic determinant, part, domain or stretch of amino acid residues on the target or antigen to which an amino acid sequence or polypeptide of the invention can bind such that the target or antigen (and/or any pathway, interaction, signalling, biological mechanism or biological effect in which the target or antigen is involved) is modulated (as defined herein).
r) An amino acid sequence or polypeptide is said to be “specific for” a first target or antigen compared to a second target or antigen when is binds to the first antigen with an affinity (as described above, and suitably expressed as a K_{D} value, K_{A} value, K_{off} rate and/or Kₒₙ rate) that is at least 10 times, such as at least 100 times, and preferably at least 1000 times, and up to 10,000 times or more better than the affinity with which said amino acid sequence or polypeptide binds to the second target or polypeptide. For example, the first antigen may bind to the target or antigen with a K_{D} value that is at least 10 times less, such as at least 100 times less, and preferably at least 1000 times less, such as 10,000 times less or even less than that, than the K_{D} with which said amino acid sequence or polypeptide binds to the second target or polypeptide. Preferably, when an amino acid sequence or polypeptide is “specific for” a first target or antigen compared to a second target or antigen, it is directed against (as defined herein) said first target or antigen, but not directed against said second target or antigen.
s) The terms “cross-block”, “cross-blocked” and “cross-blocking” are used interchangeably herein to mean the ability of an amino acid sequence or other binding agents (such as a Nanobody, polypeptide or compound or construct of the invention) to interfere with the binding of other amino acid sequences or binding agents of the invention to a given target. The extend to which an amino acid sequence or other binding agents of the invention is able to interfere with the binding of another to target, and therefore whether it can be said to cross-block according to the invention, can be determined using competition binding assays. One particularly suitable quantitative cross-blocking assay uses a Biacore machine which can measure the extent of interactions using surface plasmon resonance technology. Another suitable quantitative cross-blocking assay uses an ELISA-based approach to measure competition between amino acid sequences or other binding agents in terms of their binding to the target. The following generally describes a suitable Biacore assay for determining whether an amino acid sequence or other binding agent cross-blocks or is capable of cross-blocking according to the invention. It will be appreciated that the assay can be used with any of the amino acid sequences or other binding agents described herein. The Biacore machine (for example the Biacore 3000) is operated in line with the manufacturer's recommendations. Thus in one cross-blocking assay, the target protein is coupled to a CM5 Biacore chip using standard amine coupling chemistry to generate a surface that is coated with the target. Typically 200-800 resonance units of the target would be coupled to the chip (an amount that gives easily measurable levels of binding but that is readily saturable by the concentrations of test reagent being used). Two test amino acid sequences (termed A* and B*) to be assessed for their ability to cross-block each other are mixed at a one to one molar ratio of binding sites in a suitable buffer to create the test mixture. When calculating the concentrations on a binding site basis the molecular weight of an amino acid sequence is assumed to be the total molecular weight of the amino acid sequence divided by the number of target binding sites on that amino acid sequence. The concentration of each amino acid sequence in the test mix should be high enough to readily saturate the binding sites for that amino acid sequence on the target molecules captured on the Biacore chip. The amino acid sequences in the mixture are at the same molar concentration (on a binding basis) and that concentration would typically be between 1.00 and 1.5 micromolar (on a binding site basis). Separate solutions containing A* alone and B* alone are also prepared. A* and B* in these solutions should be in the same buffer and at the same concentration as in the test mix. The test mixture is passed over the target-coated Biacore chip and the total amount of binding recorded. The chip is then treated in such a way as to remove the bound amino acid sequences without damaging the chip-bound target. Typically this is done by treating the chip with 30 mM HCl for 60 seconds. The solution of A* alone is then passed over the target-coated surface and the amount of binding recorded. The chip is again treated to remove all of the bound amino acid sequences without damaging the chip-bound target. The solution of B* alone is then passed over the target-coated surface and the amount of binding recorded. The maximum theoretical binding of the mixture of A* and B* is next calculated, and is the sum of the binding of each amino acid sequence when passed over the target surface alone. If the actual recorded binding of the mixture is less than this theoretical maximum then the two amino acid sequences are cross-blocking each other. Thus, in general, a cross-blocking amino acid sequence or other binding agent according to the invention is one which will bind to the target in the above Biacore cross-blocking assay such that, during the assay and in the presence of a second amino acid sequence or other binding agent of the invention, the recorded binding is between 80% and 0.1% (e.g. 80% to 4%) of the maximum theoretical binding, specifically between 75% and 0.1% (e.g. 75% to 4%) of the maximum theoretical binding, and more specifically between 70% and 0.1% (e.g. 70% to 4%) of maximum theoretical binding (as just defined above) of the two amino acid sequences or binding agents in combination. The Biacore assay described above is a primary assay used to determine if amino acid sequences or other binding agents cross-block each other according to the invention. On rare occasions particular amino acid sequences or other binding agents may not bind to target coupled via amine chemistry to a CM5 Biacore chip (this usually occurs when the relevant binding site on target is masked or destroyed by the coupling to the chip). In such cases cross-blocking can be determined using a tagged version of the target, for example a N-terminal His-tagged version. In this particular format, an anti-His amino acid sequence would be coupled to the Biacore chip and then the His-tagged target would be passed over the surface of the chip and captured by the anti-His amino acid sequence. The cross blocking analysis would be carried out essentially as described above, except that after each chip regeneration cycle, new His-tagged target would be loaded back onto the anti-His amino acid sequence coated surface. In addition to the example given using N-terminal His-tagged target, C-terminal His-tagged target could alternatively be used. Furthermore, various other tags and tag binding protein combinations that are known in the art could be used for such a cross-blocking analysis (e.g. HA tag with anti-HA antibodies; FLAG tag with anti-FLAG antibodies; biotin tag with streptavidin).
   The following generally describes an ELISA assay for determining whether an amino acid sequence or other binding agent directed against a target cross-blocks or is capable of cross-blocking as defined herein. It will be appreciated that the assay can be used with any of the amino acid sequences (or other binding agents such as polypeptides of the invention) described herein. The general principal of the assay is to have an amino acid sequence or binding agent that is directed against the target coated onto the wells of an ELISA plate. An excess amount of a second, potentially cross-blocking, anti-target amino acid sequence is added in solution (i.e. not bound to the ELISA plate). A limited amount of the target is then added to the wells. The coated amino acid sequence and the amino acid sequence in solution compete for binding of the limited number of target molecules. The plate is washed to remove excess target that has not been bound by the coated amino acid sequence and to also remove the second, solution phase amino acid sequence as well as any complexes formed between the second, solution phase amino acid sequence and target. The amount of bound target is then measured using a reagent that is appropriate to detect the target. An amino acid sequence in solution that is able to cross-block the coated amino acid sequence will be able to cause a decrease in the number of target molecules that the coated amino acid sequence can bind relative to the number of target molecules that the coated amino acid sequence can bind in the absence of the second, solution phase, amino acid sequence. In the instance where the first amino acid sequence, e.g. an Ab-X, is chosen to be the immobilized amino acid sequence, it is coated onto the wells of the ELISA plate, after which the plates are blocked with a suitable blocking solution to minimize non-specific binding of reagents that are subsequently added. An excess amount of the second amino acid sequence, i.e. Ab-Y, is then added to the ELISA plate such that the moles of Ab-Y target binding sites per well are at least 10 fold higher than the moles of Ab-X target binding sites that were used, per well, during the coating of the ELISA plate. Target is then added such that the moles of target added per well are at least 25-fold lower than the moles of Ab-X target binding sites that were used for coating each well. Following a suitable incubation period the ELISA plate is washed and a reagent for detecting the target is added to measure the amount of target specifically bound by the coated anti[target amino acid sequence (in this case Ab-X). The background signal for the assay is defined as the signal obtained in wells with the coated amino acid sequence (in this case Ab-X), second solution phase amino acid sequence (in this case Ab-Y), target buffer only (i.e. without target) and target detection reagents. The positive control signal for the assay is defined as the signal obtained in wells with the coated amino acid sequence (in this case Ab-X), second solution phase amino acid sequence buffer only (i.e. without second solution phase amino acid sequence), target and target detection reagents. The ELISA assay may be run in such a manner so as to have the positive control signal be at least 6 times the background signal. To avoid any artefacts (e.g. significantly different affinities between Ab-X and Ab-Y for the target) resulting from the choice of which amino acid sequence to use as the coating amino acid sequence and which to use as the second (competitor) amino acid sequence, the cross-blocking assay may to be run in two formats: 1) format 1 is where Ab-X is the amino acid sequence that is coated onto the ELISA plate and Ab-Y is the competitor amino acid sequence that is in solution and 2) format 2 is where Ab-Y is the amino acid sequence that is coated onto the ELISA plate and Ab-X is the competitor amino acid sequence that is in solution. Ab-X and Ab-Y are defined as cross-blocking if, either in format 1 or in format 2, the solution phase anti-target amino acid sequence is able to cause a reduction of between 60% and 100%, specifically between 70% and 100%, and more specifically between 80% and 100%, of the target detection signal {i.e. the amount of target bound by the coated amino acid sequence) as compared to the target detection signal obtained in the absence of the solution phase anti-target amino acid sequence (i.e. the positive control wells).
t) An amino acid sequence is said to be “cross-reactive” for two different antigens or antigenic determinants (such as serum albumin from two different species of mammal, such as human serum albumin and cyno serum albumin) if it is specific for (as defined herein) both these different antigens or antigenic determinants.
u) By binding that is “essentially independent of the pH” is generally meant herein that the association constant (K_{A}) of the amino acid sequence with respect to the serum protein (such as serum albumin) at the pH value(s) that occur in a cell of an animal or human body (as further described herein) is at least 5%, such as at least 10%, preferably at least 25%, more preferably at least 50%, even more preferably at least 60%, such as even more preferably at least 70%, such as at least 80% or 90% or more (or even more than 100%, such as more than 110%, more than 120% or even 130% or more, or even more than 150%, or even more than 200%) of the association constant (K_{A}) of the amino acid sequence with respect to the same serum protein at the pH value(s) that occur outside said cell. Alternatively, by binding that is “essentially independent of the pH” is generally meant herein that the k_{off} rate (measured by Biacore) of the amino acid sequence with respect to the serum protein (such as serum albumin) at the pH value(s) that occur in a cell of an animal or human body (as e.g. further described herein, e.g. pH around 5.5, e.g. 5.3 to 5.7) is at least 5%, such as at least 10%, preferably at least 25%, more preferably at least 50%, even more preferably at least 60%, such as even more preferably at least 70%, such as at least 80% or 90% or more (or even more than 100%, such as more than 110%, more than 120% or even 130% or more, or even more than 150%, or even more than 200%) of the k_{off} rate of the amino acid sequence with respect to the same serum protein at the pH value(s) that occur outside said cell, e.g. pH 7.2 to 7.4. By “the pH value(s) that occur in a cell of an animal or human body” is meant the pH value(s) that may occur inside a cell, and in particular inside a cell that is involved in the recycling of the serum protein. In particular, by “the pH value(s) that occur in a cell of an animal or human body” is meant the pH value(s) that may occur inside a (sub)cellular compartment or vesicle that is involved in recycling of the serum protein (e.g. as a result of pinocytosis, endocytosis, transcytosis, exocytosis and phagocytosis or a similar mechanism of uptake or internalization into said cell), such as an endosome, lysosome or pinosome.
v) As further described herein, the total number of amino acid residues in a Nanobody can be in the region of 110-120, is preferably 112-115, and is most preferably 113. It should however be noted that parts, fragments, analogs or derivatives (as further described herein) of a Nanobody are not particularly limited as to their length and/or size, as long as such parts, fragments, analogs or derivatives meet the further requirements outlined herein and are also preferably suitable for the purposes described herein;
w) As further described in paragraph q) on pages 58 and 59 of WO 08/020,079 (incorporated herein by reference), the amino acid residues of a Nanobody are numbered according to the general numbering for V_{H} domains given by Kabat et al. (“Sequence of proteins of immunological interest”, US Public Health Services, NIH Bethesda, Md., Publication No. 91), as applied to V_{HH} domains from Camelids in the article of Riechmann and Muyldermans, J. Immunol. Methods 2000 Jun. 23; 240 (1-2): 185-195 (see for example FIG. 2 of this publication), and accordingly FR1 of a Nanobody comprises the amino acid residues at positions 1-30, CDR1 of a Nanobody comprises the amino acid residues at positions 31-35, FR2 of a Nanobody comprises the amino acids at positions 36-49, CDR2 of a Nanobody comprises the amino acid residues at positions 50-65, FR3 of a Nanobody comprises the amino acid residues at positions 66-94, CDR3 of a Nanobody comprises the amino acid residues at positions 95-102, and FR4 of a Nanobody comprises the amino acid residues at positions 103-113.
x) The Figures, Sequence Listing and the Experimental Part/Examples are only given to further illustrate the invention and should not be interpreted or construed as limiting the scope of the invention and/or of the appended claims in any way, unless explicitly indicated otherwise herein.
y) As further described herein, an anti-platelet agent or anti-platelet drug limits the migration or aggregation of blood platelets in an animal, e.g. human.
z) As further described herein, a thrombolytic agent or thrombolytic drug acts to dissolve blood clots after they have formed.
aa) As further described herein, an antithrombotic drug or agent is a drug which reduces thrombus formation.
bb) An anti vWF agent is an agent such as e.g. an antibody, single domain antibody, dAbs, or Nanobody or constructs and fragments thereof that specifically binds to von Willebrand Factor (vWF), e.g. human vWF (SEQ ID NO: 23), wherein SEQ ID NO: 23 is the following amino acid sequence:

“MIPARFAGVLLALALILPGTLCAEGTRGRSSTARCSLFGSDFVNTFDGS
MYSFAGYCSYLLAGGCQKRSFSIIGDFONGKRVSLSVYLGEFFDIHLFVN
GTVTQGDQRVSMPYASKGLYLETEAGYYKLSGEAYGFVARIDGSGNFQVL
LSDRYFNKTCGLCGNFNIFAEDDFMTQEGTLTSDPYDFANSWALSSGEQW
CERASPPSSSCNISSGEMQKGLWEQCQLLKSTSVFARCHPLVDPEPFVAL
CEKTLCECAGGLECACPALLEYARTCAQEGMVLYGWTDHSACSPVCPAGM
EYRQCVSPCARTCQSLHINEMCQERCVDGCSCPEGQLLDEGLCVESTECP
CVHSGKRYPPGTSLSRDCNTCICRNSQWICSNEECPGECLVTGQSHFKSF
DNRYFTFSGICQYLLARDCQDHSFSIVIETVQCADDRDAVCTRSVTVRLP
GLHNSLVKLKHGAGVAMDGQDIQLPLLKGDLRIQHTVTASVRLSYGEDLQ
MDWDGRGRLLVKLSPVYAGKTCGLCGNYNGNQGDDFLTPSGLAEPRVEDF
GNAWKLHGDCQDLQKQHSDPCALNPRMTRFSEEACAVLTSPTFEACHRAV
SPLPYLANCRYDVCSCSDGRECLCGALASYAAACAGRGVRVAWREPGRCE
LNCPKGQVYLQCGTPCNLTCRSLSYPDEECNEACLEGCFCPPGLYMDERG
DCVPKAQCPCYYDGEIFQPEDIFSDHHTMCYCEDGFMHCTMSGVPGSLLP
DAVLSSPLSHRSKRSLSCRPPMVKLVCPADNLRAEGLECTKTCQNYDLEC
MSMGCVSGCLCPPGMVRHENRCVALERCPCFHQGKEYAPGETVKIGCNTC
VCRDRKWNCTDHVCDATCSTIGMAHYLTFDGLKYLFPGECQYVLVQDYCG
SNPGTFRILVGNKGCSHPSVKCKKRVTILVEGGEIELFDGEVNVKRPMKD
ETHFEVVESGRYIILLLGKALSVVWDRHLSISVVLKQTYQEKVCGLCGNF
DGIQNNDLISSNLQVEEDPVDFGNSWKVSSQCADTRKVPLDSSPATCHNN
IMKQTMVDSSCRILTSDVFQDCNKLVDPEPYLDVCIYDTCSCESIGDCAC
FCDTIAAYAHVCAQHGKWTWRTATLCPQSCEERNLRENGYECEWRYNSCA
PACQVTCQHPEPLACPVQCVEGCHAHCPPGKILDELLQTCVDPEDCPVCE
VAGRRFASGKKVTLNPSDPEHCQICHCDVVNLTCEACQEPGGLVVPPTDA
PVSPTTLYVEDISEPPLHDFYCSRLLDLVFLLDGSSRLSEAEFEVLKAFV
VDMMERLRISQKWVRVAWEYHDGSHAYIGLKDRKRPSELRRIASQVKYAG
SQVASTSEVLKYTLFQIFSKIDRPEASRIALLLMASQEPQRMSRNFVRYV
QGKKKKVIVIPVGIGPHANLKQIRLIEKQAPENKAFVLSSVDELEQQRDE
IVSYLCDLAPEAPPPTLPPHMAQVTVGPGLRNSMVLDVAFVLEGSDKIGE
ADFNRSKEFMEEVIQRMDVGQDSIHVTVLQYSYMVTVEYPFSEAQSKGDI
LQRVREIRYQGGNRTNTGLALRYLSDHSFLVSQGDREQAPNLVYMVTGNP
ASDEIKRLPGDIQWPIGVGPNANVQELERIGWPNAPILIQDFETLPREAP
DLVLQRCCSGEGLQIPTLSPAPDCSOPLDVILLLDGSSSFPASYFDEMKS
FAKAFISKANIGPRLTQVSVLQYGSITTIDVPWNVVPEKAHLLSLVDVMQ
REGGPSQIGDALGFAVRYLTSEMHGARPGASKAVVILVTDVSVDSVDAAA
DAARSNRVTVFPIGIGDRYDAAQLRILAGPAGDSNVVKLQRIEDLPTMVT
LGNSFLHKLCSGFVRICMDEDGNEKRPGDVWTLPDQCHTVTCQPDGQTLL
KSHRVNCDRGLRPSCPNSQSPVKVEETCGCRWTCPCVCTGSSTRHIVTFD
GQNFKLTGSCSYVLFQNKEQDLEVILHNGACSPGARQGCMKSIEVKHSAL
SVELHSDMEVTVNGRLVSVPYVGGNMEVNVYGAIMHEVRFNHLGHIFTFT
PQNNEFQLQLSPKTFASKTYGLCGICDENGANDFMLRDGTVTTDWKTLVQ
EWTVQRPGQTCQPILEEQCLVPDSSHCQVLLLPLFAECHKVLAPATFYAI
CQQDSCHQEQVCEVIASYAHLCRTNGVCVDWRTPDFCAMSCPPSLVYNHC
EHGCPRHCDGNVSSCGDHPSEGCFCPPDKVMLEGSCVPEEACTQCIGEDG
VQHQFLEAWVPDHQPCQICTCLSGRKVNCTTQPCPTAKAPTCGLCEVARL
RQNADQCCPEYECVCDPVSCDLPPVPHCERGLQPTLTNPGECRPNFTCAC
RKEECKRVSPPSCPPHRLPTLRKTQCCDEYECACNCVNSTVSCPLGYLAS
TATNDCGCTTTTCLPDKVCVHRSTIYPVGQFWEEGCDVCTCTDMEDAVMG
LRVAQCSQKPCEDSCRSGFTYVLHEGECCGRCLPSACEVVTGSPRGDSQS
SWKSVGSQWASPENPCLINECVRVKEEVFIQQRNVSCPQLEVPVCPSGFQ
LSCKTSACCPSCRCERMEACMLNGTVIGPGKTVMIDVCTTCRCMVQVGVI
SGFKLECRKTTCNPCPLGYKEENNTGECCGRCLPTACTIQLRGGQIMTLK
RDETLQDGCDTHFCKVNERGEYFWEKRVTGCPPFDEHKCLAEGGKIMKIP
GTCCDTCEEPECNDITARLQYVKVGSCKSEVEVDIHYCQGKCASKAMYSI
DINDVQDQCSCCSPTRTEPMQVALHCTNGSVVYHEVLNAMECKCSPRKCS
K”
or
GenBank reference: NM_000552.

cc) Thromboembolism or thromboembolic disorders are disorders that are caused by the formation of a clot (thrombus) in the blood vessel that breaks loose and is carried by the blood stream to plug another vessel. The clot may plug a vessel in the lungs (pulmonary embolism), brain (stroke), gastrointestinal tract, kidneys, or leg. Thromboembolism or a thromboembolic disorder is an important cause of morbidity (disease) and mortality (death), especially in adults.
dd) For a further general description of Nanobodies®, reference is made to the prior art cited herein, such as e.g. described in WO 08/020,079 (page 16). [Note: Nanobody®, Nanobodies® and Nanoclone® are registered trademarks of Ablynx N. V.]

### 2.) Treatments of the Invention

The present invention provides a method for the treatment or use in the treatment of a thromboembolic disorder such as e.g. a myocardial infarction, ischemic stroke, deep vein thrombosis or pulmonary embolism, preferably ischemic stroke such as acute ischemic stroke; in patients, preferably humans, in need thereof, wherein said treatment comprises administering
i) an effective dose regimen of an anti vWF agent, e.g. an A1 vWF binding agent, a vWF binding agent with the epitope of 12a2h1, a selected vWF binding agent (any of SEQ ID NO: 1 to 18 or single domain antibody such as e.g. a nanobody having a CDR combination as shown in any of SEQ ID NO: 1 to 18) or ALX-0081 (SEQ ID NO: 1); and
ii) a low dose regimen of a thrombolytic agent, e.g. such as rtPA, to said patient; and
   wherein optionally the time point when the anti vWF agent and thrombolytic agent is administered is later than indicated in the case where an anti-thrombolytic agent is administered alone, e.g. later than the standard of care limit of 3 hours or shorter for a standard of care dose of rt-PA (or e.g. later than the standard of care limit of a 6 h or shorter within the event for a standard of care dose of rt-PA administered on site).

An effective dose regimen of an anti vWF agent, e.g. an A1 vWF binding agent, a vWF binding agent with the epitope of 12a2h1, a selected vWF binding agent (any of SEQ ID NO: 1 to 18 or single domain antibody such as e.g. a nanobody having a CDR combination as shown in any of SEQ ID NO: 1 to 18) or ALX-0081 (SEQ ID NO: 1); is a dose regimen that is able to reduce the ex vivo maximum aggregation below 10% measured by RIPA or below 20% RICO activity measured by RICO (RIPA, ristocetin induced platelet aggregation—(Favaloro E J. Clin Haematol 2001; 14: 299-319.), RICO, Ristocetin Cofactor Platelet Agglutination Assay—(Howard M A, Firkin B G. Ristocetin—a new tool in the investigation of platelet aggregation. Thrombosis et Diathesis Haemorrhagica 1971; 26: 362-9) upon administration of compound see also WO 2009/115614. An example for an effective dose regimen for ALX-0081 in humans, such as e.g. humans with acute coronary syndrome, is a multiple dose, intravenous dose of ALX-0081 every 6 h for 24 h starting with 6 mg and 3 times 4 mg but may be also a dose range such as e.g. 2 to 16 mg ALX-0081 every 6 h (e.g. for 24 h) or simply a dose of ALX-0081 (such as e.g. 16 mg of ALX-0081) wherein the interval of application of the next dose is guided by monitoring the RIPA, i.e. RIPA is not higher than 10% or by monitoring RICO, i.e. RICO is not higher than 20%.

A low dose regimen of a thrombolytic agent is a dose regimen that is known to the skilled person in the art. For example, low dose rtPA protocols have been used that utilizes pulse spray injection of rtPA directly into the thrombus in a total amount of 4 mg or less of rtPA each day for thrombolytic therapy (see e.g. Low-Dose rtPA to Treat Blood Clots in Major Arm or Neck Veins (sponsored by NIHCC) clinical trials.gov identifier is NCT00055159). However, a low dose may also be any dose that is a dose per day that is less than the standard or care that is about 1 to 1.5 mg/kg/per day.

The particular dosage regimen may be further influenced by the attending physician taking into account the particulars of the patient, especially age, weight, life style, activity level, and general medical condition as appropriate.

Moreover, the present invention provides a method for the treatment or use in the treatment of a thromboembolic disorder such as e.g. a myocardial infarction, ischemic stroke, deep vein thrombosis or pulmonary embolism, preferably ischemic stroke such as acute ischemic stroke; in patients, preferably humans, in need thereof, wherein said treatment comprises the inhibition of reocclusion in said patients treated with a thrombolytic agent, e.g. such as rtPA, by administering an effective dose regimen of an anti vWF agent, e.g. an A1 vWF binding agent, a vWF binding agent with the epitope of 12a2h1, a selected vWF binding agent (any of SEQ ID NO: 1 to 18 or single domain antibody such as e.g. a nanobody having a CDR combination as shown in any of SEQ ID NO: 1 to 18) or ALX-0081 (SEQ ID NO: 1).

Moreover, the present invention provides a method for the treatment or use in the treatment of a thromboembolic disorder such as e.g. a myocardial infarction, ischemic stroke, deep vein thrombosis or pulmonary embolism, preferably ischemic stroke such as acute ischemic stroke; in patients, preferably humans, in need thereof, wherein said patients has rtPA resistant thrombi, and wherein said treatment comprises the administration of an effective dose regimen of an anti vWF agent, e.g. an A1 vWF binding agent, a vWF binding agent with the epitope of 12a2h1, a selected vWF binding agent (any of SEQ ID NO: 1 to 18 or single domain antibody such as e.g. a nanobody having a CDR combination as shown in any of SEQ ID NO: 1 to 18) or ALX-0081 (SEQ ID NO: 1).

Equivalent uses, combinations and pharmaceutical compositions related to the anti vWF agent and thrombolytic agent as outlined in the method above and herein are also provided.

The invention further provides a vWF binding agent with the epitope of 12a2h1, wherein said agent is not an agent that is a nanobody or comprises a nanobody having identical CDRs from any of the nanobodies 12a2 (SEQ ID NO:20), 12a5 (SEQ ID NO:21), and/or 12b6 (SEQ ID NO:22); and wherein said binding agent with the epitope of 12a2h1 has an epitope on the A1 domain of the vWF that consists of at least 1 atom in a sphere of 3 Angstrom or more around the 12a2h1-vWF binding site, i.e. wherein said binding agent with the epitope of 12a2h1 interacts at least with the following A1-vWF amino acid residues that are at the positions 500, 502, 503, 505-511, 545 and 550 of the A1 domain of the vWF (taking into account the numbering of the A1 domain of vWF as set out in Cruz et al. Journal of Biological Chemistry (2000), 275 (25), 19098-19105), more preferably wherein said binding agent with the epitope of 12a2h1 has an epitope on the A1 domain of the vWF that consists of at least 1 atom in a sphere of 4 Angstrom or more around the 12a2h1-vWF binding site, i.e. wherein said binding agent with the epitope of 12a2h1 interacts at least with the following A1-vWF amino acid residues that are at the positions 498, 500, 502-511, 545, 550, 695 and 701 of the A1 domain of the vWF (taking into account the numbering of the A1 domain of vWF as set out in Cruz et al. Journal of Biological Chemistry (2000), 275 (25), 19098-19105), even more preferably wherein said binding agent with the epitope of 12a2h1 has an epitope on the A1 domain of the vWF that consists of at least 1 atom in a sphere of 5 Angstrom or more around the 12a2h1-vWF binding site, i.e. wherein said binding agent with the epitope of 12a2h1 interacts at least with the following A1-vWF amino acid residues that are at the positions 498, 500-511, 545, 550, 692, 695, 696, 700 and 701 of the A1 domain of the vWF (taking into account the numbering of the A1 domain of vWF as set out in Cruz et al, Journal of Biological Chemistry (2000), 275 (25), 19098-19105), more preferably wherein said binding agent with the epitope of 12a2h1 has an epitope on the A1 domain of the vWF that consists of at least 1 atom in a sphere of 6 Angstrom or more around the 12a2h1-vWF binding site, i.e. wherein said binding agent with the epitope of 12a2h1 interacts at least with the following A1-vWF amino acid residues that are at the positions 498, 500-511, 543, 545, 550, 691, 692, 695, 696, 700 and 701 of the A1 domain of the vWF (taking into account the numbering of the A1 domain of vWF as set out in Cruz et al. Journal of Biological Chemistry (2000), 275 (25), 19098-19105).

The invention yet further provides an in vitro screening method for the generation of the nanobodies of the invention using the epitope information described in this invention. Generally, it should be noted that the term nanobody as used herein in its broadest sense is not limited to a specific biological source or to a specific method of preparation. For example, the nanobodies of the invention can generally be obtained by any of the techniques (1) to (8) mentioned on pages 61 and 62 of WO 08/020,079, or any other suitable technique known per se. One preferred class of nanobodies corresponds to the V_{HH} domains of naturally occurring heavy chain antibodies directed against the epitope of 12a2h1 on vWF as defined herein. Such naturally occurring V_{HH} domains against the epitope of 12a2h1 on vWF as defined herein, can be obtained from naïve libraries of Camelid V_{HH} sequences, for example by screening such a library using the epitope of 12a2h1 on vWF as defined herein, using one or more screening techniques known per se. For example, the invention yet further provides an in vitro screening method by screening such a library using the above described epitope using one or more screening techniques known per se. Such libraries and techniques are for example described in WO 99/37681, WO 01/90190, WO 03/025020 and WO 03/035694. Alternatively, improved synthetic or semi-synthetic libraries derived from naïve V_{HH} libraries may be used, such as V_{HH} libraries obtained from naïve V_{HH} libraries by techniques such as random mutagenesis and/or CDR shuffling, as for example described in WO 00/43507.

Thus, in another aspect, the invention relates to a method for generating nanobodies that are directed against the epitope of 12a2h1 on vWF as defined herein. In one aspect, said method at least comprises the steps of:
a) providing a set, collection or library of nanobody sequences; and
b) screening said set, collection or library of Nanobody sequences for Nanobody sequences that can bind to and/or have affinity for the epitope of 12a2h1 on vWF as defined herein;
   and
c) isolating the Nanobody or Nanobodies that can bind to and/or have affinity for the epitope of 12a2h1 on vWF as defined herein.

In such a method, the set, collection or library of nanobody sequences may be a naïve set, collection or library of nanobody sequences; a synthetic or semi-synthetic set, collection or library of nanobody sequences; and/or a set, collection or library of nanobody sequences that have been subjected to affinity maturation.

In a preferred aspect of this method, the set, collection or library of nanobody sequences may be an immune set, collection or library of nanobody sequences, and in particular an immune set, collection or library of V_{HH} sequences, that have been derived from a species of Camelid that has been suitably immunized with the epitope of 12a2h1 on vWF as defined herein. In one particular aspect, said epitope of 12a2h1 on vWF as defined herein may be embedded in an antigenic determinant region.

In the above methods, the set, collection or library of nanobody or V_{HH} sequences may be displayed on a phage, phagemid, ribosome or suitable micro-organism (such as yeast), such as to facilitate screening. Suitable methods, techniques and host organisms for displaying and screening (a set, collection or library of) Nanobody sequences will be clear to the person skilled in the art, for example on the basis of the further disclosure herein. Reference is also made to WO 03/054016 and to the review by Hoogenboom in Nature Biotechnology, 23, 9, 1105-1116 (2005).

In another aspect, the method for generating Nanobody sequences comprises at least the steps of:
a) providing a collection or sample of cells derived from a species of Camelid that express immunoglobulin sequences;
b) screening said collection or sample of cells for (i) cells that express an immunoglobulin sequence that can bind to and/or have affinity for the epitope of 12a2h1 on vWF as defined herein; and (ii) cells that express heavy chain antibodies, in which substeps (i) and (ii) can be performed essentially as a single screening step or in any suitable order as two separate screening steps, so as to provide at least one cell that expresses a heavy chain antibody that can bind to and/or has affinity for the epitope of 12a2h1 on vWF as defined herein; and
c) either (i) isolating from said cell the V_{HH} sequence present in said heavy chain antibody; or (ii) isolating from said cell a nucleic acid sequence that encodes the V_{HH} sequence present in said heavy chain antibody, followed by expressing said V_{HH} domain.

In the method according to this aspect, the collection or sample of cells may for example be a collection or sample of B-cells. Also, in this method, the sample of cells may be derived from a Camelid that has been suitably immunized with the epitope of 12a2h1 on vWF as defined herein.

The above method may be performed in any suitable manner, as will be clear to the skilled person. Reference is for example made to EP 0 542 810, WO 05/19824, WO 04/051268 and WO 04/106377. The screening of step b) is preferably performed using a flow cytometry technique such as FACS. For this, reference is for example made to Lieby et al., Blood, Vol. 97, No. 12, 3820. Particular reference is made to the so-called “Nanoclone™” technique described in International application WO 06/079372 by Ablynx N.V.

In another aspect, the method for generating an amino acid sequence directed against the epitope of 12a2h1 on vWF as defined herein may comprise at least the steps of:
a) providing a set, collection or library of nucleic acid sequences encoding heavy chain antibodies or Nanobody sequences;
b) screening said set, collection or library of nucleic acid sequences for nucleic acid sequences that encode a heavy chain antibody or a nanobody sequence that can bind to and/or has affinity for the epitope of 12a2h1 on vWF as defined herein;
   and
c) isolating said nucleic acid sequence, followed by expressing the V_{HH} sequence present in said heavy chain antibody or by expressing said nanobody sequence, respectively.

In such a method, the set, collection or library of nucleic acid sequences encoding heavy chain antibodies or nanobody sequences may for example be a set, collection or library of nucleic acid sequences encoding a naïve set, collection or library of heavy chain antibodies or V_{HH} sequences; a set, collection or library of nucleic acid sequences encoding a synthetic or semi-synthetic set, collection or library of nanobody sequences; and/or a set, collection or library of nucleic acid sequences encoding a set, collection or library of nanobody sequences that have been subjected to affinity maturation.

In a preferred aspect of this method, the set, collection or library of nucleic acid sequences may be an immune set, collection or library of nucleic acid sequences encoding heavy chain antibodies or V_{HH} sequences derived from a Camelid that has been suitably immunized with the epitope of 12a2h1 on vWF as defined herein.

In the above methods, the set, collection or library of nucleotide sequences may be displayed on a phage, phagemid, ribosome or suitable micro-organism (such as yeast), such as to facilitate screening. Suitable methods, techniques and host organisms for displaying and screening (a set, collection or library of) nucleotide sequences encoding amino acid sequences will be clear to the person skilled in the art, for example on the basis of the further disclosure herein. Reference is also made to WO 03/054016 and to the review by Hoogenboom in Nature Biotechnology, 23, 9, 1105-1116 (2005).

As will be clear to the skilled person, the screening step of the methods described herein can also be performed as a selection step. Accordingly the term “screening” as used in the present description can comprise selection, screening or any suitable combination of selection and/or screening techniques. Also, when a set, collection or library of sequences is used, it may contain any suitable number of sequences, such as 1, 2, 3 or about 5, 10, 50, 100, 500, 1000, 5000, 10⁴, 10⁵, 10⁶, 10⁷, 10⁸ or more sequences.

Also, one or more or all of the sequences in the above set, collection or library of amino acid sequences may be obtained or defined by rational or semi-empirical approaches such as computer modelling techniques or biostatics or data mining techniques.

Furthermore, such a set, collection or library can comprise one, two or more sequences that are variants from one another (e.g. with designed point mutations or with randomized positions), compromise multiple sequences derived from a diverse set of naturally diversified sequences (e.g. an immune library), or any other source of diverse sequences (as described for example in Hoogenboom et al, Nat Biotechnol 23:1105, 2005 and Binz et al, Nat Biotechnol 2005, 23:1247). Such set, collection or library of sequences can be displayed on the surface of a phage particle, a ribosome, a bacterium, a yeast cell, a mammalian cell, and linked to the nucleotide sequence encoding the amino acid sequence within these carriers. This makes such set, collection or library amenable to selection procedures to isolate the desired amino acid sequences of the invention. More generally, when a sequence is displayed on a suitable host or host cell, it is also possible (and customary) to first isolate from said host or host cell a nucleotide sequence that encodes the desired sequence, and then to obtain the desired sequence by suitably expressing said nucleotide sequence in a suitable host organism. Again, this can be performed in any suitable manner known per se, as will be clear to the skilled person.

Furthermore, such an amino acid sequence such as e.g. a nanobody directed against the epitope of 12a2h1 on vWF as defined herein may not include an agent that is a nanobody or comprises a nanobody having identical CDRs from any of the nanobodies 12a2 (SEQ ID NO:20), 12a5 (SEQ ID NO:21), and/or 12b6 (SEQ ID NO:22).

The uses and methods of the present invention represent an improvement to existing therapy of thromboembolic disorders in which a combination of i) an anti vWF agent, e.g. an A1 vWF binding agent, a vWF binding agent with the epitope of 12a2h1, a selected vWF binding agent (any of SEQ ID NO: 1 to 18 or single domain antibody such as e.g. a nanobody having a CDR combination as shown in any of SEQ ID NO: 1 to 18) or ALX-0081 (SEQ ID NO: 1); and ii) an thrombolytic agent are used to inhibit inappropriate thrombus formation and to reduce the already formed inappropriate thrombus or clot in the blood vessels of patients with said disorders.

Thus in the present description the terms “treatment” or “treat” refer to both prophylactic or preventative treatment as well as curative or palliative treatment of inappropriate thrombus formation under high shear condition and include not only new formation of thrombus but also reduction of the thrombus. The terms “treatment” or “treat” refer especially in the treatment setting in patients with a thromboembolic disorder or having a risk to develop a thromboembolic disorder such as e.g. a myocardial infarction, ischemic stroke, deep vein thrombosis or pulmonary embolism.

Thus in the present description the terms “prevent”, “preventing” and “prevention” (and the like) include, in addition to complete prevention, “reduce”, “reducing”, “reduction”, “inhibit”, “inhibiting” and “inhibition” of inappropriate thrombus formation under high shear condition and reduction of existing clots or thrombi.

Thus in a particular embodiment, the invention provides:
i) a method for the treatment of a thromboembolic disorder;
ii) a pharmaceutical composition for the treatment of a thromboembolic disorder; or
iii) the use in the treatment of a thromboembolic disorder,
   a. wherein said treatment comprises administering to a patient:
      i. an effective dose regimen of an anti vWF agent; and
      ii. a low dose regimen of a thrombolytic agent; and
   b. wherein optionally the time point when the specific anti vWF agent and thrombolytic agent is administered can be expanded beyond standard care; and
   c. wherein optionally the anti vWF agent is an agent selected from the group consisting of an A1 vWF binding agent, a vWF binding agent with the epitope of 12a2h1, a selected vWF binding agent (any of SEQ ID NO: 1 to 18 or single domain antibody such as e.g. a nanobody having a CDR combination as shown in any of SEQ ID NO: 1 to 18) and ALX-0081 (SEQ ID NO: 1) and wherein said selected agent is able to prevent of thrombus formation under high shear condition at a concentration of 1 ug/ml or less, preferably 0.5 ug/ml or less, e.g. is able to inhibit ristocetin or shear-induced platelet aggregation (such as shown e.g. in example 16 of WO2004/062551) at a concentration of 1 ug/ml or less, preferably 0.5 ug/ml or less; and
   d. wherein optionally the thrombolytic agent is rtPA; and
   e. wherein optionally said thromboembolic disorder is a disorder selected from the group consisting of myocardial infarction, ischemic stroke, deep vein thrombosis and pulmonary embolism, preferably ischemic stroke such as acute ischemic stroke.

The specific A1 vWF binding agents used in the present invention are typically those which prevent thrombus formation under high shear condition, in particular those which are indicated to have a safe application in patients with a thromboembolic disorder, e.g. a disorder selected from the group consisting of myocardial infarction, ischemic stroke, deep vein thrombosis and pulmonary embolism, preferably ischemic stroke such as acute ischemic stroke.

Thus, for example, suitable agents of specific A1 vWF binders for use in the invention may include the compounds in Table 1 or a compound having 80% or more, more preferably 85% or more, most preferred 90%, 95%, 96%, 97%, 98%, 99% or more, amino acid sequence identity to a compound in Table A-2 (see Definition section for “sequence identity”).

In another preferred selection, suitable agents of specific A1 vWF binders for use in the invention may include agents such as e.g. antibodies that cross-block or are cross-blocked by the compounds of Table 1 (see Definition section for “cross-blocked” and “cross-block”). In another preferred selection, suitable agents of specific A1 vWF binders for use according to the present invention are antibodies, preferably single variable domains, cross-blocking at least 50% of ALX-0081 (SEQ ID NO: 1) binding, more preferably at least 60%, more preferably at least 70%, even more preferably at least 80% of ALX-0081 binding. In another preferred selection, suitable agents of specific A1 vWF binders for use according to the present invention are antibodies, preferably single variable domains, cross-blocked at least 50% by ALX-0081 (SEQ ID NO: 1), more preferably at least 60%, more preferably at least 70%, even more preferably at least 80% by ALX-0081. Said cross-blocking or cross-blocked measurements are e.g. done by BiaCore measurements.

TABLE 1
Examples of specific A1 vWF binders

| | SEQ | |
|---|---|---|
| | ID | |
| Name | NO | Sequence |
| 12a2h1-3a- | 1 | EVQLVESGGGLVQPGGSLRLSCAASGRTFSYNPMGWFRQAPG |
| 12a2h1 (ALX- | | KGRELVAAISRTGGSTYYPDSVEGRFTISRDNAKRMVYLQMNSL |
| 0081) | | RAEDTAVYYCAAAGVRAEDGRVRTLPSEYTFWGQGTQVTVSSA |
| | | AAEVQLVESGGGLVQPGGSLRLSCAASGRTESYNPMGWFRQAP |
| | | GKGRELVAAISRTGGSTYYPDSVEGRFTISRDNAKRMVYLQMNS |
| | | LRAEDTAVYYCAAAGVRAEDGRVRTLPSEYTFWGQGTQVTVSS |
| 12A2-3a-12A2 | 2 | QVKLEESGGGLVQAGGALRLSCAASGRTESYNPMGWFRQAPG |
| | | KERDLVAAISRTGGSTYYPDSVEGRFTISRDNAKRMVYLQMNNL |
| | | KPEDTAVYYCAAAGVRAEDGRVRTLPSEYTFWGQGTQVTVSSA |
| | | AAEVQLVESGGGLVQAGGALRLSCAASGRTFSYNPMGWFRQAP |
| | | GKERDLVAAISRTGGSTYYPDSVEGRFTISRDNAKRMVYLQMNN |
| | | LKPEDTAVYYCAAAGVRAEDGRVRTLPSEYTFWGQGTQVTVSS |
| 12A2-GS9-12A2 | 3 | QVKLEESGGGLVQAGGALRLSCAASGRTFSYNPMGWFRQAPG |
| | | KERDLVAAISRTGGSTYYPDSVEGRFTISRDNAKRMVYLQMNNL |
| | | KPEDTAVYYCAAAGVRAEDGRVRTLPSEYTFWGQGTQVTVSSG |
| | | GGGSGGGSEVQLVESGGGLVQAGGALRLSCAASGRTFSYNPM |
| | | GWFRQAPGKERDLVAAISRTGGSTYYPDSVEGRFTISRDNAKRM |
| | | VYLQMNNLKPEDTAVYYCAAAGVRAEDGRVRTLPSEYTFWGQG |
| | | TQVTVSS |
| 12A2-GS30-12A2 | 4 | QVKLEESGGGLVQAGGALRLSCAASGRTFSYNPMGWFRQAPG |
| | | KERDLVAAISRTGGSTYYPDSVEGRFTISRDNAKRMVYLQMNNL |
| | | KPEDTAVYYCAAAGVRAEDGRVRTLPSEYTFWGQGTQVTVSSG |
| | | GGGSGGGGSGGGGSGGGGSGGGGSGGGGSEVQLVESGGGL |
| | | VQAGGALRLSCAASGRTFSYNPMGWFRQAPGKERDLVAAISRT |
| | | GGSTYYPDSVEGRFTISRDNAKRMVYLQMNNLKPEGTAVYYCAA |
| | | AGVRAEDGRVRTLPSEYTFWGQGTQVTVSS |
| 12A5-3a-12A5 | 5 | AVQLVESGGGLVQPGGSLRLSCLASGRIFSIGAMGMYRQAPGK |
| | | QRELVATITSGGSTNYADPVKGRFTISRDGPKNTVYLQMNSLKPE |
| | | DTAVYYCYANLKQGSYGYRENDYWGQGTQVTVSSAAAEVQLVE |
| | | SGGGLVQPGGSLRLSCLASGRIFSIGAMGMYRQAPGKQRELVAT |
| | | ITSGGSTNYADPVKGRFTISRDGPKNTVYLOMNSLKPEDTAVYYC |
| | | YANLKQGSYGYRENDYWGQGTQVTVSS |
| 12A5-GS9-12A5 | 6 | AVQLVESGGGLVQPGGSLRLSCLASGRIFSIGAMGMYRQAPGK |
| | | QRELVATITSGGSTNYADPVKGRFTISRDGPKNTVYLQMNSLKPE |
| | | DTAVYYCYANLKQGSYGYRFNDYWGQGTQVTVSSGGGGSGGG |
| | | SEVQLVESGGGLVQPGGSLRLSCLASGRIFSIGAMGMYRQAPGK |
| | | QRELVATITSGGSTNYADPVKGRFTISRDGPKNTVYLQMNSLKPE |
| | | DTAVYYCYANLKQGSYGYRFNDYWGQGTQVTVSS |
| 12A5-GS30-12A5 | 7 | AVQLVESGGGLVQPGGSLRLSCLASGRIFSIGAMGMYRQAPGK |
| | | QRELVATITSGGSTNYADPVKGRFTISRDGPKNTVYLQMNSLKPE |
| | | DTAVYYCYANLKQGSYGYRENDYWGQGTQVTVSSGGGGSGGG |
| | | GSGGGGSGGGGSGGGGSGGGGSEVQLVESGGGLVQPGGSLR |
| | | LSCLASGRIFSIGAMGMYRQAPGKQRELVATITSGGSTNYADPVK |
| | | GRFTISRDGPKNTVYLQMNSLKPEDTAVYYCYANLKQGSYGYRF |
| | | NDYWGQGTQVTVSS |
| 12B6-3a-12B6 | 8 | QVQLVESGGGLVQAGGALRLSCAASGRTFSYNPMGWFRQAPG |
| | | KERDVVAAISRTGGSTYYARSVEGRFTISRDNAKRMVYLQMNAL |
| | | KPEDTAVYYCAAAGVRAEDGRVRTLPSEYNFWGQGTQVTVSSA |
| | | AAEVQLVESGGGLVQAGGALRLSCAASGRTFSYNPMGWFRQAP |
| | | GKERDVVAAISRTGGSTYYARSVEGRFTISRDNAKRMVYLQMNA |
| | | LKPEDTAVYYCAAAGVRAEDGRVRTLPSEYNFWGQGTQVTVSS |
| 12B6-GS9-12B6 | 9 | QVQLVESGGGLVQAGGALRLSCAASGRTFSYNPMGWFRQAPG |
| | | KERDVVAAISRTGGSTYYARSVEGRFTISRDNAKRMVYLQMNAL |
| | | KPEDTAVYYCAAAGVRAEDGRVRTLPSEYNFWGQGTQVTVSSG |
| | | GGGSGGGSEVQLVESGGGLVQAGGALRLSCAASGRTFSYNPM |
| | | GWFRQAPGKERDVVAAISRTGGSTYYARSVEGRFTISRDNAKRM |
| | | VYLQMNALKPEDTAVYYCAAAGVRAEDGRVRTLPSEYNFWGQG |
| | | TQVTVSS |
| 12B6-GS30-12B6 | 10 | QVQLVESGGGLVQAGGALRLSCAASGRTFSYNPMGWFRQAPG |
| | | KERDVVAAISRTGGSTYYARSVEGRFTISRDNAKRMVYLQMNAL |
| | | KPEDTAVYYCAAAGVRAEDGRVRTLPSEYNFWGQGTQVTVSSG |
| | | GGGSGGGGSGGGGSGGGGSGGGGSGGGGSEVQLVESGGGL |
| | | VQAGGALRLSCAASGRTFSYNPMGWFRQAPGKERDVVAAISRT |
| | | GGSTYYARSVEGRFTISRDNAKRMVYLQMNALKPEDTAVYYCAA |
| | | AGVRAEDGRVRTLPSEYNFWGQGTQVTVSS |
| 12A2H4-3a- | 11 | EVQLVESGGGLVQPGGSLRLSCAASGRTESYNPMGWFRQAPG |
| 12A2H4 | | KGRELVAAISRTGGSTYYPDSVEGRFTISRDNAKRSVYLQMNSL |
| | | RAEDTAVYYCAAAGVRAEDGRVRTLPSEYTFWGQGTQVTVSSA |
| | | AAEVQLVESGGGLVQPGGSLRLSCAASGRTFSYNPMGWFRQAP |
| | | GKGRELVAAISRTGGSTYYPDSVEGRFTISRDNAKRSVYLQMNS |
| | | LRAEDTAVYYCAAAGVRAEDGRVRTLPSEYTFWGQGTQVTVSS |
| 12B6H2-3a- | 12 | EVQLVESGGGLVQPGGSLRLSCAASGRTFSYNPMGWFRQAPG |
| 12B6H2 | | KGREVVAAISRTGGSTYYARSVEGRFTISRDNAKRMVYLQMNSL |
| | | RAEDTAVYYCAAAGVRAEDGRVRTLPSEYNFWGQGTQVTVSSA |
| | | AAEVOLVESGGGLVQPGGSLRLSCAASGRTESYNPMGWFRQAP |
| | | GKGREVVAAISRTGGSTYYARSVEGRFTISRDNAKRMVYLQMNS |
| | | LRAEDTAVYYCAAAGVRAEDGRVRTLPSEYNFWGQGTQVTVSS |
| 12A2H1-GS9- | 13 | EVQLVESGGGLVQPGGSLRLSCAASGRTFSYNPMGWFRQAPG |
| 12A2H1 | | KGRELVAAISRTGGSTYYPDSVEGRFTISRDNAKRMVYLQMNSL |
| | | RAEDTAVYYCAAAGVRAEDGRVRTLPSEYTFWGQGTQVTVSSG |
| | | GGGSGGGSEVQLVESGGGLVQPGGSLRLSCAASGRTFSYNPM |
| | | GWFRQAPGKGRELVAAISRTGGSTYYPDSVEGRFTISRDNAKRM |
| | | VYLQMNSLRAEDTAVYYCAAAGVRAEDGRVRTLPSEYTFWGQG |
| | | TQVTVSS |
| 12A2H4-GS9- | 14 | EVQLVESGGGLVQPGGSLRLSCAASGRTFSYNPMGWFRQAPG |
| 12A2H4 | | KGRELVAAISRTGGSTYYPDSVEGRFTISRDNAKRSVYLQMNSL |
| | | RAEDTAVYYCAAAGVRAEDGRVRTLPSEYTFWGQGTQVTVSSG |
| | | GGGSGGGSEVQLVESGGGLVQPGGSLRLSCAASGFITFSYNPM |
| | | GWFRQAPGKGRELVAAISRTGGSTYYPDSVEGRFTISRDNAKRS |
| | | VYLQMNSLRAEDTAVYYCAAAGVRAEDGRVRTLPSEYTFWGQG |
| | | TQVTVSS |
| 12B6H2-GS9- | 15 | EVQLVESGGGLVQPGGSLRLSCAASGRTFSYNPMGWFRQAPG |
| 12B6H2 | | KGREVVAAISRTGGSTYYARSVEGRFTISRDNAKRMVYLQMNSL |
| | | RAEDTAVYYCAAAGVRAEDGRVRTLPSEYNFWGQGTQVTVSSG |
| | | GGGSGGGSEVQLVESGGGLVQPGGSLRLSCAASGRTFSYNPM |
| | | GWFRQAPGKGREVVAAISRTGGSTYYARSVEGRFTISRDNAKR |
| | | MVYLQMNSLRAEDTAVYYCAAAGVRAEDGRVRTLPSEYNFWGQ |
| | | GTQVTVSS |
| 2A2H1-GS30- | 16 | EVQLVESGGGLVQPGGSLRLSCAASGRTFSYNPMGWFRQAPG |
| 12A2H1 | | KGRELVAAISRTGGSTYYPDSVEGRFTISRDNAKRMVYLQMNSL |
| | | RAEDTAVYYCAAAGVRAEDGRVRTLPSEYTFWGQGTQVTVSSG |
| | | GGGSGGGGSGGGGSGGGGSGGGGSGGGGSEVQLVESGGGL |
| | | VQPGGSLRLSCAASGRTFSYNPMGWFRQAPGKGRELVAAISRT |
| | | GGSTYYPDSVEGRFTISRDNAKRMVYLQMNSLRAEDTAVYYCAA |
| | | AGVRAEDGRVRTLPSEYTFWGQGTQVTVSS |
| 12A2H4-G530- | 17 | EVQLVESGGGLVQPGGSLRLSCAASGRTFSYNPMGWFRQAPG |
| 12A2H4 | | KGRELVAAISRTGGSTYYPDSVEGRFTISRDNAKRSVYLQMNSL |
| | | RAEDTAVYYCAAAGVRAEDGRVRTLPSEYTFWGQGTQVTVSSG |
| | | GGGSGGGGSGGGGSGGGGSGGGGSGGGGSEVQLVESGGGL |
| | | VQPGGSLRLSCAASGRTFSYNPMGWFRQAPGKGRELVAAISRT |
| | | GGSTYYPDSVEGRFTISRDNAKRSVYLQMNSLRAEDTAVYYCAA |
| | | AGVRAEDGRVRTLPSEYTFWGQGTQVTVSS |
| 12B6H2-GS30- | 18 | EVQLVESGGGLVQPGGSLRLSCAASGRTFSYNPMGWFRQAPG |
| 12B6H2 | | KGREVVAAISRTGGSTYYARSVEGRFTISRDNAKRMVYLQMNSL |
| | | RAEDTAVYYCAAAGVRAEDGRVRTLPSEYNFWGQGTQVTVSSG |
| | | GGGSGGGGSGGGGSGGGGSGGGGSGGGGSEVQLVESGGGL |
| | | VQPGGSLRLSCAASGRTFSYNPMGWFRQAPGKGREVVAAISRT |
| | | GGSTYYARSVEGRFTISRDNAKRMVYLQMNSLRAEDTAVYYCAA |
| | | AGVRAEDGRVRTLPSEYNFWGQGTQVTVSS |
| 12A2h1 | 19 | EVQLVESGGGLVQPGGSLRLSCAASGRTFSYNPMGWFRQAPG |
| | | KGRELVAAISRTGGSTYYPDSVEGRFTISRDNAKRMVYLQMNSL |
| | | RAEDTAVYYCAAAGVRAEDGRVRTLPSEYTFWGQGTQVTVSS |
| 12A2 | 20 | QVKLEESGGGLVQAGGALRLSCAASGRTFSYNPMGWFRQAPG |
| | | KERDLVAAISRTGGSTYYPDSVEGRFTISRDNAKRMVYLQMNNL |
| | | KPEDTAVYYCAAAGVRAEDGRVRTLPSEYTFWGQGTQVTVSS |
| 12A5 | 21 | AVQLVESGGGLVQPGGSLRLSCLASGRIFSIGAMGMYRQAPGK |
| | | QRELVATITSGGSTNYADPVKGRFTISRDGPKNTVYLQMNSLKPE |
| | | DTAVYYCYANLKQGSYGYRFNDYWGQGTQVTVSS |
| 12B6 | 22 | QVQLVESGGGLVQAGGALRLSCAASGRTFSYNPMGWFRQAPG |
| | | KERDVVAAISRTGGSTYYARSVEGRFTISRDNAKRMVYLQMNAL |
| | | KPEDTAVYYCAAAGVRAEDGRVRTLPSEYNFWGQGTQVTVSS |

Preferably the specific A1 vWF binders for use in the invention are the 12a2h1-like compounds. For the purposes of the present description a 12a2h1-like compound is a compound which comprises 12a2h1 (i.e. SEQ ID NO: 19) or a compound having 80% or more, more preferably 85% or more, most preferred 90%, 95%, 96%, 97%, 98%, 99% or more, amino acid sequence identity to 12a2h1 (SEQ ID NO: 19): A particularly preferred specific A1 vWF binder is ALX-0081 (SEQ ID NO: 1).

All the specific A1 vWF binders mentioned above are well known from the literature. This includes their manufacture (see in particular e.g. WO 2006/122825 but also WO 2004/062551). For example, ALX-0081 is prepared as described e.g. in WO 2006/122825.

The vWF binding agent with an epitope to 12a2h1 that is identical or overlapping to the nanobody 12a2h1 (SEQ ID NO: 19) is a binding agent that has an epitope on the A1 domain of the vWF that consists of at least 1 atom in a sphere of 3 Angstrom or more around the 12a2h1-vWF binding site, i.e. wherein said binding agent with the epitope of 12a2h1 interacts at least with the following A1-vWF amino acid residues that are at the positions 500, 502, 503, 505-511, 545 and 550 of the A1 domain of the vWF (taking into account the numbering of the A1 domain of vWF as set out in Cruz et al. Journal of Biological Chemistry (2000), 275 (25), 19098-19105), more preferably wherein said binding agent with the epitope of 12a2h1 has an epitope on the A1 domain of the vWF that consists of at least 1 atom in a sphere of 4 Angstrom or more around the 12a2h1-vWF binding site, i.e. wherein said binding agent with the epitope of 12a2h1 interacts at least with the following A1-vWF amino acid residues that are at the positions 498, 500, 502-511, 545, 550, 695 and 701 of the A1 domain of the vWF (taking into account the numbering of the A1 domain of vWF as set out in Cruz et al. Journal of Biological Chemistry (2000), 275 (25), 19098-19105), even more preferably wherein said binding agent with the epitope of 12a2h1 has an epitope on the A1 domain of the vWF that consists of at least 1 atom in a sphere of 5 Angstrom or more around the 12a2h1-vWF binding site, i.e. wherein said binding agent with the epitope of 12a2h1 interacts at least with the following A1-vWF amino acid residues that are at the positions 498, 500-511, 545, 550, 692, 695, 696, 700 and 701 of the A1 domain of the vWF (taking into account the numbering of the A1 domain of vWF as set out in Cruz et al. Journal of Biological Chemistry (2000), 275 (25), 19098-19105), more preferably wherein said binding agent with the epitope of 12a2h1 has an epitope on the A1 domain of the vWF that consists of at least 1 atom in a sphere of 6 Angstrom or more around the 12a2h1-vWF binding site, i.e. wherein said binding agent with the epitope of 12a2h1 interacts at least with the following A1-vWF amino acid residues that are at the positions 498, 500-511, 543, 545, 550, 691, 692, 695, 696, 700 and 701 of the A1 domain of the vWF (taking into account the numbering of the A1 domain of vWF as set out in Cruz et al. Journal of Biological Chemistry (2000), 275 (25), 19098-19105).

The thrombolytic agent may be an agent such as e.g. a tissue plasminogen activator (herein also referred to as “t-PA, rt-PA, rtPA, Alteplase, alteplase activase”), a reteplase (herein also referred to as “retavase”), a tenecteplase (herein also referred to as “TNKase”), an anistreplase (herein also referred to as “Eminase”), a streptokinase (herein also referred to as “Kabikinase, Streptase”), and/or an urokinase (herein also referred to as “Abbokinase”).

The specific vWF agents as disclosed herein and specific thrombolytic agents as disclosed herein (hereinafter referred to also as the Agents of the Invention) may be used in the form of a polypeptide concentrate or ready-to-use solution (hereinafter also referred to as “pharmaceutical composition of the invention”). For example, the Agents of the Invention can be used in a pharmaceutical composition comprising a buffer (such as e.g. citrate, histidine, Tris, PBS, d-PBS), a tonicifier (such as e.g. mannitol, glycine or sodium chloride) and a surfactant (such as e.g. Polysorbate 80 or Polysorbate 20). Additionally, osmolytes and preservatives may be added. The Agents of the Invention may be in a small-volume, high-dose solution such as e.g. in an amount of from 1 mg agent per ml solution up to 100 mg, e.g. 2 to 50 mg agent per ml solution. Other concentrations such as e.g. 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95 mg per ml solution are also feasible.

A preferred pharmaceutical formulation for ALX-0081 comprises between 1 to 20 mg, e.g. 5 or 10 mg, ALX-0081 per ml solution that comprises a buffer, a tonicifier and a surfactant. A more preferred pharmaceutical composition comprises between 1 to 20 mg, e.g. 5 or 10 mg, ALX-0081 per ml solution that consists of a buffer, e.g. d-PBS, a tonicifier, e.g. glycine, and a surfactant, e.g. Polysorbate 80. An even more preferred pharmaceutical composition comprises 5 (+/−1) mg/ml ALX-0081, suitable d-PBS buffer; suitable amount of glycine; and a suitable amount of Polysorbate 80 pH 7.1. A most preferred pharmaceutical composition comprises 5 (+/−1) mg/ml ALX-0081, 0.137 M NaCl, 3.7 mM KH₂ PO₄, 9.8 mM Na₂ HPO₄x2H₂O, 2.7 KCl, 0.2 M glycine, 0.02% (volume %) Polysorbate 80 pH 7.1. Said compositions may be in the form of a concentrate and thus e.g. the dose applied to a patient in need thereof may be adopted by diluting the concentrate to the desired dose (see e.g. experimental part for suitable doses).

A preferred pharmaceutical formulation for rtPA (e.g. 100 mg rtPA) comprises L-Arginine (e.g. 3.5 mg per 100 mg rtPA), phosphoric acid (e.g. 1 mg per 100 mg rtPA), polysorbate 80 (approximately 11 mg per 100 mg rtPA) and sterile water.

The Agents of the invention are preferably used in the form of pharmaceutical compositions that contain a therapeutically appropriate (as described herein) amount of active ingredient optionally together with or in admixture with inorganic or organic, solid or liquid, pharmaceutically acceptable carriers which are suitable for administration.

The pharmaceutical compositions may be, for example, compositions for oral, pulmonary, or parenteral administration, more preferably parenteral administration, such as intravenous or subcutaneous administration, or compositions for transdermal administration (e.g. passive or iontophoretic).

Preferably, the pharmaceutical compositions are adapted to parenteral (especially intravenous, intra-arterial or transdermal) administration. Intravenous administration is considered to be of particular importance. Preferably the Agents of the invention are in the form of a parenteral form, most preferably an intravenous or subcutaneous form.

The particular mode of administration and the dosage may be selected by the attending physician taking into account the particulars of the patient, especially age, weight, life style, activity level, and general medical condition as appropriate.

However, in general the dosage of the Agents of the Invention may depend on various factors, such as effectiveness and duration of action of the active ingredient, warm-blooded species, and/or sex, age, weight and individual condition of the warm-blooded animal.

Formulations in single dose unit form contain preferably from about 1 to about 20 mg, e.g. 5 mg/ml and formulations not in single dose unit form contain preferably from also about 1 to about 20 mg, e.g. 5 mg/ml of the active ingredient.

Pharmaceutical preparations for parenteral administration are, for example, those in dosage unit forms, such as ampoules. They are prepared in a manner known per se, for example by means of conventional mixing, dissolving or lyophilising processes.

Parenteral formulations are especially injectable fluids that are effective in various manners, intra-arterially, intramuscularly, intraperitoneally, intranasally, intradermally, subcutaneously or preferably intravenously and subcutaneously. Such fluids are preferably isotonic aqueous solutions or suspensions which can be prepared before use, for example from lyophilised preparations or concentrate which contain the active ingredient alone or together with a pharmaceutically acceptable carrier. The pharmaceutical preparations may be sterilised and/or contain adjuncts, for example preservatives, stabilisers, wetting agents and/or emulsifiers, solubilisers, salts for regulating the osmotic pressure and/or buffers.

Suitable formulations for transdermal application include an effective amount of the active ingredient with carrier. Advantageous carriers include absorbable pharmacologically acceptable solvents to assist passage through the skin of the host. Characteristically, transdermal devices are in the form of a bandage comprising a backing member, a reservoir containing the compound optionally with carriers, optionally a rate controlling barrier to deliver the active ingredient of the skin of the host at a controlled and predetermined rate over a prolonged period of time, and means to secure the device to the skin.

The following Experimental Part illustrates the invention described hereinbefore.

### EXPERIMENTAL PART

### Example 1

### Photochemical-Induced Middle Cerebral Artery (MCA) Occlusion Model in Guinea Pigs

### Material and Methods

### Materials

TABLE A-1

| | | Catalog | Concentration, |
|---|---|---|---|
| Materials | Provider | number | Formulation |
| Rose Bengal | Sigma Aldrich | R3877 | NA |
| rtPA | Boehringer | RVG | 20 mg (lyophilized), |
| (Actilyse ®) | Ingelheim | 12247 UR | reconstituted in 20 |
| | (IP International | | mL sterile water |
| | Pharmacy GmbH) | | (provided) |
| ALX-0081 | Ablynx | | 5.205 mg/mL, |
| | | | DPBS pH 7.1 + |
| | | | 0.2M glycine + |
| | | | 0.05% Tween-80 |
| Hartley-Dunkey | Charles River, | NA | NA |
| Guinea pig | Italy | | |
| Drabkin reagent | Sigma Aidrich | D5941 | NA |
| Laser dopler | Transonic | ABLPHN20 | NA |
| probe | | | |

### Methods

### PK/PD Study

The aim of this study was to analyze in Hartley-Dunkey guinea pigs the pharmacokinetics (PK) and pharmacodynamics (PD) of ALX-0081. The PD of ALX-0081 can be measured via the ristocetin-induced platelet aggregation (RIPA) technique and its equivalent ristocetin cofactor (RICO) assay. Both techniques are accepted clinically and measure the ability of ristocetin-activated vWF to interact with the platelet receptor GP1b-IX-V. We wanted to have a dosing regimen of ALX-0081 which gives inhibition in the RICO assay for 24 h, without using infusion pumps. Hartley-Dunkey guinea pigs, male (50%) and female (50%), weighing about 400-450 g (Charles River, Italy) were used in this study. The animals were numbered and divided in groups of 3 individuals.

The dosing regimen was simulated based on the results of a previous PK/PD study, where the plasma PK profiles of ALX-0081 were compared after a single intravenous (i.v.) or subcutaneous (s.c.) administration to female guinea pigs (20 mg/kg, 7 mg/kg and 1 mg/kg for both routes). This simulation showed that with a dosing regimen of 0.2 mg/kg i.v.+0.8 mg/kg s.c. on t=0 h and 1.5 mg/kg s.c. on t=6 h (total dose of 2.5 mg/kg), a full inhibition of the RICO could be expected for approximately 24 h. In addition, a dosing scheme with a lower (1.25 mg/kg: 0.1 mg/kg i.v.+0.4 mg/kg s.c. on t=0 h+0.75 mg/kg s.c. on t=6 h) and one with a higher cumulative dose (5 mg/kg: 0.4 mg/kg i.v.+1.6 mg/kg s.c. on t=0 h and 3 mg/kg s.c. on t=8 h) of ALX-0081 was tested (Table A-2).

TABLE A-2
Dosages and sampling schedule for guinea pigs receiving ALX-0081

| | | Dose^{#} | | | | | | | | | | | | |
|---|---|---|---|---|---|---|---|---|---|---|---|---|---|---|
| # | Drug | t0 | t0 | t6 | Total | N | pre | 5′ | 30′ | 1 h | 2 h | 4 h | 6 h | 24 h |
| 1 | ALX-0081 | 0.1 | 0.4 | 0.75 | 1.25 | 3 | X | X | X | X | X | X | X | X |
| 2 | | 0.2 | 0.8 | 1.5 | 2.5 | 3 | X | X | X | X | X | X | X | X |
| 3 | | 0.4 | 1.6 | 3 | 5 | 3 | X | X | X | X | X | X | X | X |

^{#}First dose will be injected i.v. on t = 0 h, the second and third dose will be injected s.c.

Blood samples were taken at different time points for PK and PD analysis (0.5 ml per time point) through a catheter inserted into the carotid. Blood samples were collected into tubes with citrate (0.32% final concentration) anticoagulant.

### Photochemical-Induced Middle Cerebral Artery (MCA) Occlusion Model in Guinea Pigs

### Surgery

The model is according to the method of Moriguchi A et al.¹

Briefly, animals were anesthetized with ketamine and xylazine. A catheter for the administration of drugs was inserted into the left jugular vein while a catheter for rose Bengal (RB) infusion was inserted in the femoral artery. After a left temporal incision, the temporal muscle was removed. A subtemporal craniotomy was performed using a dental drill under an operation microscope to open a 6-mm-diameter oval bony window. The main trunk of the MCA was observed without cutting the dura mater. The head of a 3-mm-diameter optic fiber mounted on a micromanipulator was placed on the MCA segment proximal to the olfactory tract for photoirradiation. Blood flow velocity in the MCA was measured by a pen-type pulse-Doppler flow probe (Transonic) positioned on the MCA 2-3 mm distal to the irradiated segment. Photoirradiation was conducted using a xenon lamp (Hamamatsu Photonics, Hamamatsu, Japan) with a heat-absorption filter and a green filter. When a stable baseline blood flow was obtained, rose Bengal infusion and photoirradiation with green light (wavelength 540 nm, intensity 600,000 lux for 15 min) was simultaneously started. In the optimization experiments, different doses of rose Bengal were analyzed, namely 10 mg/kg, 20 mg/kg and 30 mg/kg, all were infused for 6 min. In subsequent experiments, 20 mg/kg rose Bengal infused over a period of 6 min was used.

The probe of the laser Doppler was gently positioned close to the vessel wall to measure the blood movements under its surface (˜1 mm³). Cerebral blood flow (CBF) was measured and results expressed as tissue perfusion units (TPU). In conditions of complete occlusion of the MCA, CBF was expected to be 12±2 TPU, a value expressing blood movements in the examined tissue outside the MCA. This “zero” value was subtracted from the values recorded for each treated animal, to standardize the analysis and to report only TPUs expressing blood flow in the vessel of interest. CBF was measured for 3 hours after the start of the operation, after which animals were allowed to recover from anesthesia. Body temperature was maintained at 36° C. by a heating pad during surgery. At the end of the photoirradiation period, the skin incision was sutured.

### Administration

Just before administration, ALX-0081 (5.205 mg/mL) was diluted in vehicle buffer (DPBS pH 7.1+0.2M glycine+0.05% Tween-80) to the appropriate concentrations. ALX-0081 was administered before or after the induction of the photochemical damage to the MCA at the lowest dosing regimen capable to inhibit the ex vivo RICO for 24 hours, namely 0.4 mg/kg i.v.+1.6 mg/kg s.c. on t=0 h and 3 mg/kg s.c. on t=8 h.

One vial of rtPA (20 mg, lyophilized powder, Boehringer Ingelheim) was reconstituted with 20 mL sterile water for injection (Boehringer Ingelheim) without preservative to make a 1 mg/mL solution. rtPA was administered after the induction of the photochemical damage to the MCA. Two dosing regimens have been analysed, namely 0.032 mg/kg (bolus)+0.576 mg/kg (infusion over 30 min) and 0.1 mg/kg (bolus)+0.9 mg/kg (infusion over 30 min). Doses of rtPA were chosen based on literature^{1,2}. In one group, ALX-0081 (0.4 mg/kg i.v.+1.6 mg/kg s.c. on t=0 h and 3 mg/kg s.c. on t=8 h) and rtPA (0.032 mg/kg as bolus+0.576 mg/kg as an infusion over 30 minutes) were administered simultaneously. Both test items were prepared as described above. In control animals, PBS was administered.

### Template Bleeding Time

To evaluate if the administration of the drugs exerts an effect on haemostasis in the guinea pig, the template bleeding time model was assessed. A standardized cut was inflicted on the ventral face of the foot of guinea pigs using a commercial bleeding time template (Surgicutt, ITC, USA). The blood emerging from the cut was blotted every 30 sec with filter paper until the arrest of bleeding and the total time to bleeding arrest was calculated. The bleeding time was measured at baseline, 30 min and 2 hours after the first administration of drugs.

### Termination and Read-Outs

Guinea pigs were sacrificed 24 hrs after the end of photoirradiation by overdose of anesthetic. For ischemic brain damage analysis, the brains were coronally sectioned using Ringer solution in the presence of oxygen (entire striatum was cut in sections of 500 μm) and were stained with 1% of 2,3,5-triphenyltetrazolium chloride (TTC, Sigma) at 37° C. for 10 min. TTC stained sections were photographed and brain damage (indicated by a white area in the damaged hemisphere) was calculated using image analysis software (Image J software) and reported as % of brain area damaged (the calculation was made considering the damaged hemisphere).

For measurement of intracerebral hemorrhage, TTC stained sections were collected and homogenized. Subsequently, supernatants were collected by centrifugation at 10,000×g for 20 min and then treated with Drabkin reagent (Sigma) for 15 min at RT to convert hemoglobin into cyanomethemoglobin. The absorbance of cyanomethemoglobin was measured at 540 nm. After transforming the absorbance data into corresponding hemoglobin levels through use of a standard curve, the degree of hemorrhage was expressed as percent increase of hemoglobin in the damaged hemisphere compared to the undamaged hemisphere.

### Results

### PK/PD Study

A PK/PD study with 3 different dosing regimens of ALX-0081 (total doses of 1.25, 2.5 and 5 mg/kg; N=3/dosing regimen) was performed with the aim to find an optimal dose of ALX-0081 which gives inhibition of the RICO for 24 h.

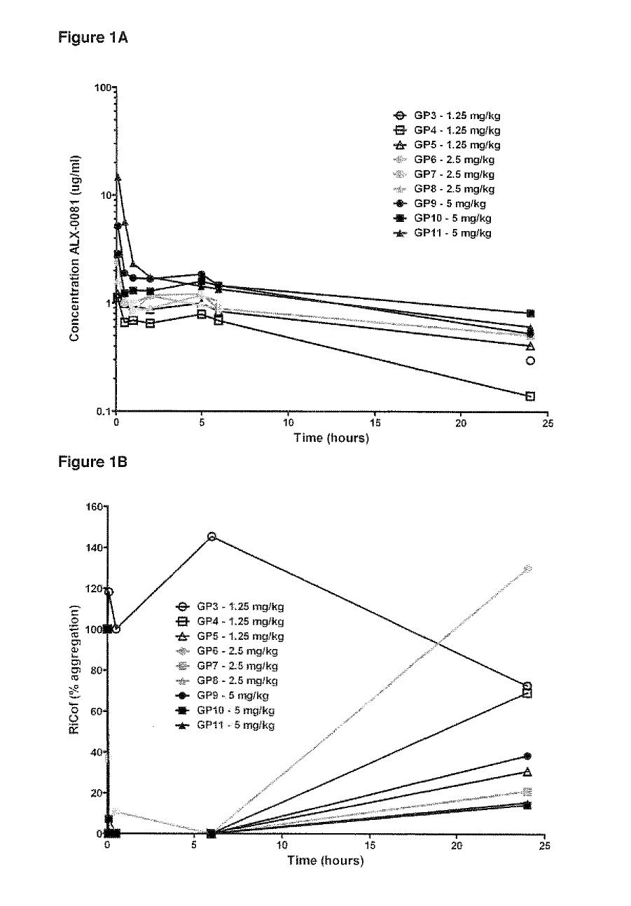

The PK profiles showed an increase in ALX-0081 plasma levels with higher administered doses (FIG. 1A). In all dose groups, plasma concentrations declined in a multiphasic manner. After an initial fast decline of the i.v. administered ALX-0081, plasma levels tend to increase again due to the s.c. administered dose and a second maximum was reached at approximately 5 hrs post iv and sc injections. Six hours after the first i.v. and s.c. administrations, an additional s.c. dose was given. The RICO results also showed a dose-dependent response. In all animals, except one animal in the lowest dose group which was incorrectly dosed, complete inhibition of the RICO was observed in the first 6 hours (FIG. 1B). After 24 hours, only the animals in the highest dose group (total dose of 5 mg/kg) showed 70-90% inhibition of this PD marker. In the other 2 dose groups, the RICO was back to basal levels in at least 1 of the 3 animals (FIG. 1B). Therefore, we concluded that only a cumulative dose of 5 mg/kg would give us complete inhibition of the RICO for 24 hours.

### Optimization of the Stroke Model

To optimize the model, a more standardized damage of the MCA was desired. In previous experiments, total occlusion of the MCA was not obtained in some of the animals with a 10-min irradiation of the MCA in combination with a 10 mg/kg dose of rose Bengal (RB) infused over 6 min. Therefore, a reduction of the blood flow under 40% of baseline as critical point was suggested, which occurred approximately 30 to 40 min after the beginning of RB infusion. By increasing the amount of RB, the damage and the time to total occlusion (CBF≦12±2 TPU) was standardized. The latter was preferably obtained in all animals in less than 30 min after the beginning of RB infusion.

Nine animals were randomized in three groups (N=3). The amount of RB in the different groups was varied. When a stable baseline blood flow was established, RB mg/kg (Group 1), 30 mg/kg (Group 2) or 50 mg/kg (Group 3) was administered as an infusion over 6 min. The MCA was irradiated for 15 min, starting simultaneously with RB infusion.

Results showed that a faster occlusion of the MCA and less variation in time to occlusion between animals was obtained with a 30 mg/kg and 50 mg/kg dose of RB compared to the 20 mg/kg dose (FIG. 2A). Mean time to occlusion was 25 min, 16 min and 13 min for the 20 mg/kg, 30 mg/kg and 50 mg/kg dose, respectively. Because the 50 mg/kg dose showed some side effects (coloring of the skin), it was concluded that the 30 mg/kg was the preferred RB dose. Analysis of the brain damage showed a good correlation with the time to occlusion. Brain damage was higher in animals in which the MCA was rapidly occluded (FIG. 2B). Brain damage was less than 5% only in these animals in which the MCA was totally occluded more than 20 min after the start of the photoirradiation.

By using drabkin reagent (Sigma) to measure hemoglobin content in the brains, we have obtained an objective way of measuring the degree of brain hemorrhage. Compatibility with TTC staining was shown. Some brains, previously injected with a known volume of blood, were first coronally sectioned and stained with 1% of 2,3,5-triphenyltetrazolium chloride (TTC). After staining, all sections were collected, homogenized and treated with Drabkin reagent. Subsequently, hemoglobin concentration was measured (data not shown).

Effect of ALX-0081 and rtPA in the Stroke Model

The aim of the study was to assess the effect of ALX-0081 and rtPA on the photochemically-induced thrombosis in the MCA of guinea pigs by evaluation of the CBF (by continuous laser Doppler measurement of blood flow), assessment of brain damage (by TTC staining) and determination of intracerebral hemorrhage (by measuring hemoglobin content). The effect of ALX-0081 and rtPA on the template bleeding time was also analyzed.

Thirty guinea pigs were randomized in 6 groups (N=5 in each group). Each of the animals was infused i.v. (via femoral artery) for 6 min with 30 mg/kg RB, immediately followed by an irradiation for 10 min. The guinea pigs from group I received vehicle. In group 2, the guinea pigs followed a dosing regimen of ALX-0081 (total dose of 5 mg/kg), starting just before the start of the photoirradiation (pre-injury). In groups 3-6, ALX-0081 and/or rtPA were administered after the induction of the photochemical damage to the MCA, starting from the moment total occlusion was obtained (CBF≦12±2 TPU; post-injury). In group 3, the guinea pigs received the same dosing regimen of ALX-0081 as group 2 (total dose of 5 mg/kg). The guinea pigs of group 4 received a combination therapy of ALX-0081 (total dose of 5 mg/kg) with rtPA (0.032 mg/kg as bolus+0.576 mg/kg as infusion), starting simultaneously from the moment total occlusion was obtained. In group 5, guinea pigs received a bolus of rtPA (0.032 mg/kg), immediately followed by a continuous infusion of rtPA for 30 min (0.576 mg/kg). Based on literature, this would be a suboptimal dose of rtPA¹. Guinea pigs of group 6 received a higher and clinically more relevant dose of rtPA, namely 0.1 mg/kg as bolus+0.9 mg/kg as infusion.

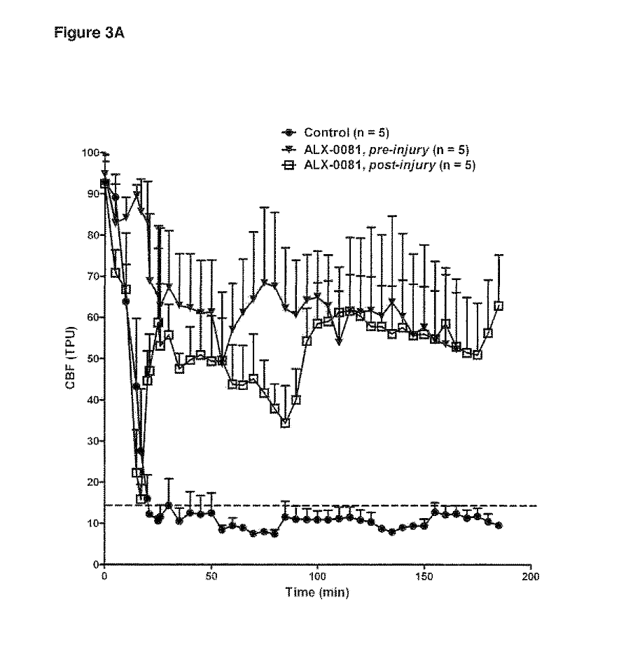

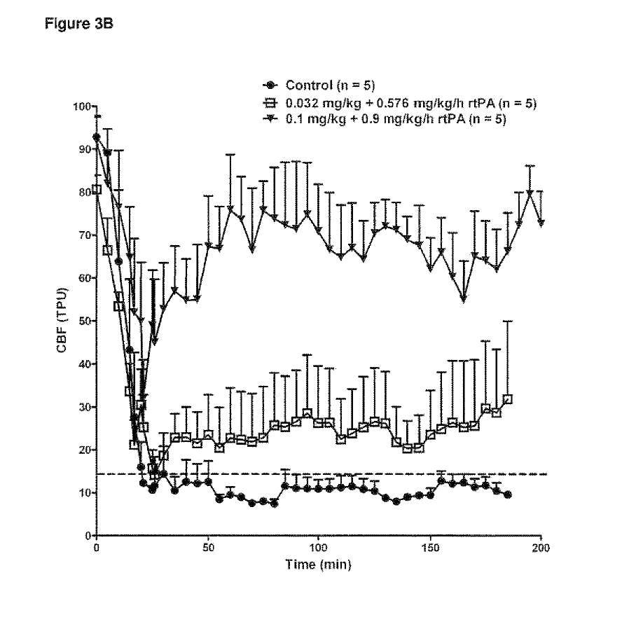

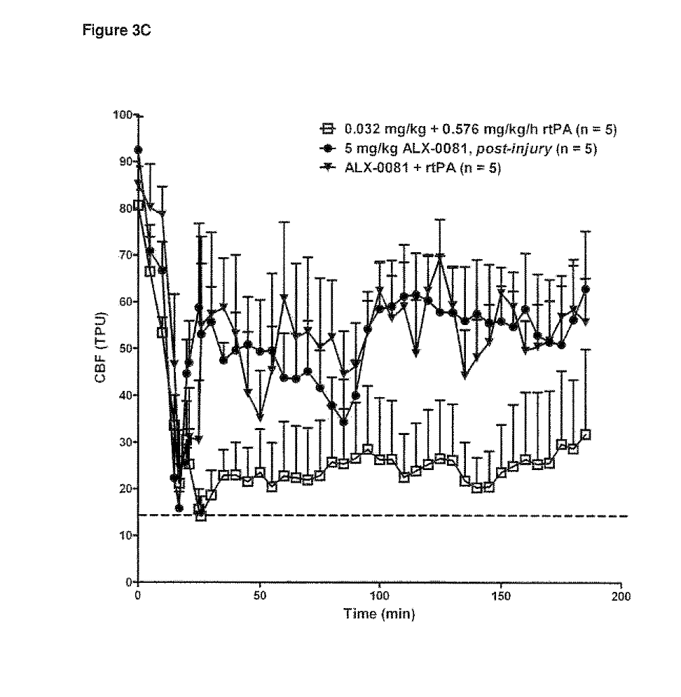

After photochemical damage, the MCA was occluded by a platelet-rich thrombus. The time required to have this occlusion of the MCA was measured by a laser Doppler probe positioned on the artery close to the site of damage. The cerebral blood flow (CBF) was measured and expressed in tissue perfusion units (TPUs). The time from the end of the photoirradiation period to occlusion (time to occlusion; CBF≦12±2 TPU) was 19±6 min in vehicle animals (FIGS. 3A&B). In guinea pigs treated with ALX-0081 pre-injury no complete occlusion of the MCA was observed (FIG. 3A). In these animals, the mean CBF did not drop below 50 TPU. In groups 3-6, ALX-0081 and/or rtPA were administered after occlusion of the MCA. In these groups, mean time to occlusion was between 15-20 minutes (FIGS. 3B&C). When ALX-0081 (total dose of 5 mg/kg) was administered after the induction of the ischemic damage, complete reperfusion of the MCA was obtained directly after treatment (FIG. 3A). This was also the case when the guinea pigs were treated with a high dose of rtPA (0.1 mg/kg as bolus+0.9 mg/kg as infusion; FIG. 3B). A low dose of rtPA (0.032 mg/kg as bolus+0.576 mg/kg as infusion), however, was sub-optimal and no complete reperfusion could be obtained in this group (FIG. 38). When this low rtPA dose was combined with ALX-0081 (total dose of 5 mg/kg), complete reperfusion of the MCA was again observed (FIG. 3C).

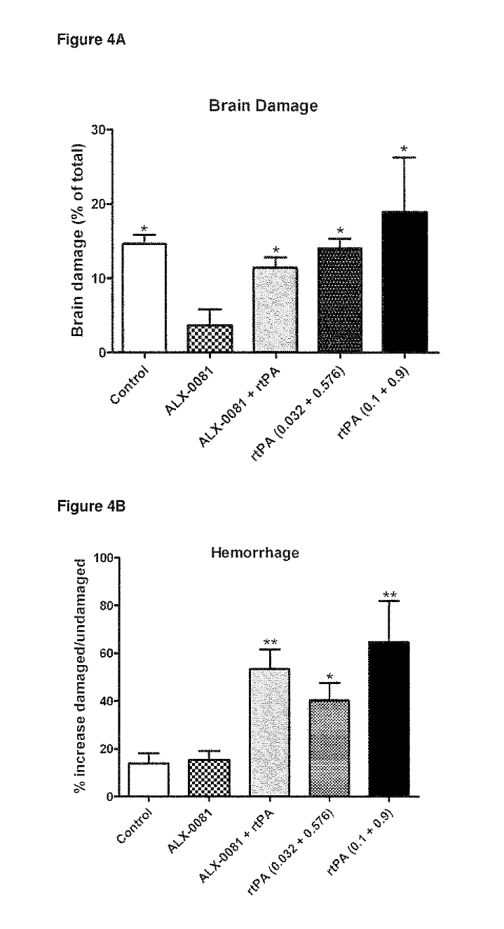

The analysis of ischemic brain damage and intracerebral hemorrhage was carried out 24 hrs after the induction of ischemia. Results are shown in FIG. 4.

In control animals, the ischemic area was 14.6±2.7% and a 13.8±9.7% increase in hemorrhage was measured. ALX-0081 (post-injury) was able to significantly reduce the ischemic area (3.6±4.8%) while no increased intracerebral bleeding was observed (15.3±8.6%). Treatment with rtPA had a dose-dependent effect on hemorrhage and brain damage. Intracerebral bleeding was increased in both rtPA-treated groups. While there was already a 40.4±14.2% increase in hemoglobin content in the low dose rtPA (0.032+0.576 mg/kg) group, this intracerebral bleeding was further increased in the high dose rtPA (0.1+0.9 mg/kg) group to 64.7%±38.8 (FIG. 4B). The sub-optimal dose of rtPA led to a comparable brain damage as in the control group (14.1±2.9%), while a high dose of rtPA even increased brain damage (15.7±10.1%), possibly due to the intracranial bleeding (FIG. 4A). The ALX-0081+rtPA combination therapy did not significantly reduce brain damage (11.4±3.1%) and intracerebral bleeding (53.4±18.4%) compared to the control and high dose rtPA groups, respectively (FIGS. 4A&B).

The template bleeding time was carried out before the procedure, 30 min after the i.v. bolus administration and 2 hrs after the beginning of the procedure.

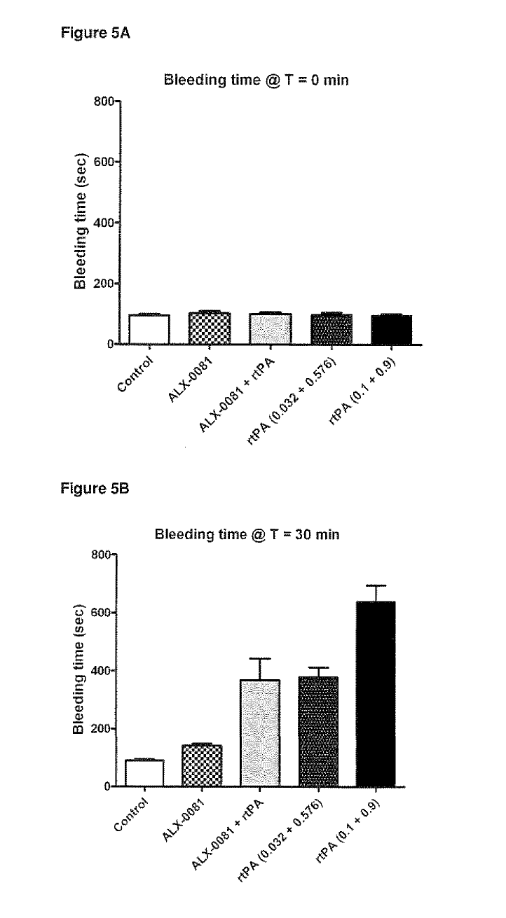

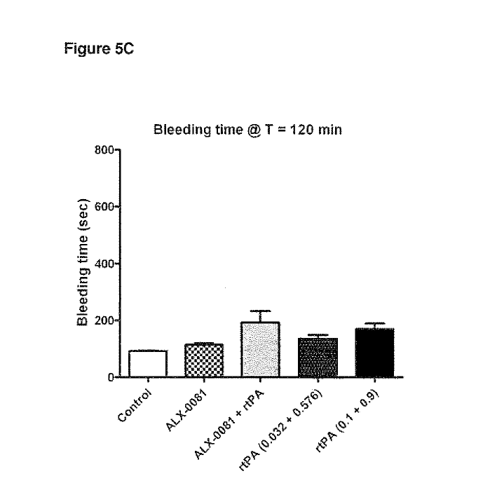

Vehicle and ALX-0081 had no effect on the template bleeding time. rtPA, however, induced a significant and dose-dependent prolongation of the template bleeding time min after bolus administration (FIG. 5B) that tended to normalize after 2 hours (FIG. 5C). This was the case for all rtPA-treated groups.

### DISCUSSION

In a first phase of the study an optimal dosing regimen of ALX-0081 was found which gave complete inhibition of the RICO for 24 hours. In this dosing scheme, one i.v. administration of 0.4 mg/kg (on t=0 h) was combined with two s.c. administrations (1.6 mg/kg on t=0 h and 3 mg/kg on t=6 h). With this new dosing regimen, the desired drug levels could be obtained without the use of infusion pumps.

The objective of the second phase of the study was to optimize the model by increasing the dose of Rose Bengal and consequently increasing the damage to the MCA. By doing this, we obtained a more reproducible time to total occlusion of the MCA and the extent of brain damage correlated well with the time to occlusion. Previously, it was also reported that brain damage correlates with the time to reperfusion and the total MCA occlusion time^{4,5}.

In addition, measurement of the hemoglobin content was evaluated as a read-out for the degree of hemorrhage in the brain. It was shown that this is a more objective method to assess intracerebral bleeding compared to macroscopically analysis of the brains. The method is compatible with the brain damage assessment by TTC staining.

Although rtPA is currently the only FDA-approved treatment for acute ischemic stroke, rtPA can only be used in limited cases due to the potential risk of brain hemorrhage and the brief 3 h time window of efficacy from symptom onset to treatment. To validate the optimized MCA thrombosis model in guinea pig and to ensure accurate comparison with ALX-0081, clinical relevant doses of rtPA were analyzed in this model. rtPA reperfused the MCA dose-dependently, suggesting that rtPA effectively lysed the obstructive thrombus in the MCA. However, rtPA also increased the degree of hemorrhage in a dose-dependent manner, leading to brain damage. The effective and safe dosages of rtPA were similar to these previously reported^{1,2}.

When administered before the injury in the optimized stroke model, ALX-0081 was effective in preventing occlusion of the MCA. If administered after the onset of ischemia, ALX-0081, as monotherapy or in combination with rtPA, was able to induce a complete reperfusion of the MCA. As ALX-0081 has no or only limited thrombolytic activity, it most likely prevented the secondary thrombus formation after spontaneous reperfusion of the MCA. Spontaneous reperfusion after the first occlusion and regeneration of occlusive platelet thrombi was already previously observed in this photochemically-induced thrombosis model. Reperfusion in combination with reocclusion has also been observed in human cerebral arteries in some patients treated with rtPA³. Therefore, inhibition of reocclusion and improvement of brain circulation by ALX-0081 is also expected to prevent development of cerebral infarction in humans. ALX-0081 not only improved the blood flow in the MCA but also ameliorated ischemic brain damage. Compared to the vehicle group, brain damage was reduced in the guinea pigs which received ALX-0081 monotherapy. The template bleeding time was also assessed and was only prolongated in the rtPA-treated groups. The hemoglobin content measurement in the brain may represent a more predictive model for the pro-hemorrhagic potential of antithrombotic agents in patients with acute ischemic stroke.

In conclusion, ALX-0081 was found to prevent reocclusion and decrease brain damage in the photochemically-induced MCA thrombosis model in guinea pig and showed a superior efficacy and safety profile in this model compared to rtPA. The fact that ALX-0081 has no effect on the incidence of hemorrhage in this model while the brain damage is reduced favors the view that ALX-0081 is a potentially promising antiplatelet agent for the treatment of acute ischemic stroke, in which intracranial hemorrhage by antithrombotic agents is the most lethal complication. Given that large platelet-rich thrombi contribute to the clinical failure of thrombolysis with rtPA (del Zoppo, 1992), ALX-0081 can be beneficial in the case of rtPA-resistant thrombi. Even if there is successful tysis of the thrombus in the major artery, downstream platelet-rich thrombus formation in the microvasculature may produce ischemic damage for which ALX-0081 therapy may show a benefit over treatment with rtPA.

### REFERENCES

¹ Moriguchi A et al. Restoration of middle cerebral artery thrombosis by novel glycoprotein IIb/IIIa antagonist FK419 in guinea pig. Eur J Pharmacol 2004; 498:179
² Mihara et al. Prohemorrhagic and bleeding time activities of recombinant tissue plasminogen activator, heparin, aspirin, and a glycoprotein IIb/IIIa antagonist. Journal of Neurotrauma. 2005; Vol 22:11
³ Alexandrov A V, Grotta J C. Arterial reocclusion in stroke patients treated with intravenous tissue plasminogen activator. Neurology. 2002; 59; 862-867
⁴ Kawano et al., Am J. Physiol. 1998; 275: 1578-1583
⁵ Kawano et al., Eur J. Pharmacol. 1999; 374: 377-385

### Example 2

### Crystal Structure of A1-vWF in Complex with the Nanobody 12a2h1

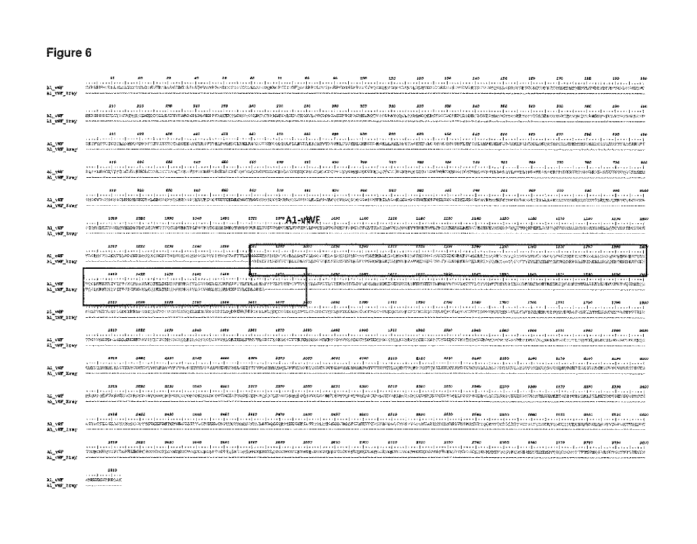

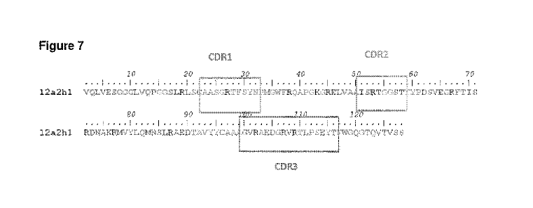

The A1-vWF domain is part of the multimeric von Willebrand Factor and the complete sequence of the protein is shown in FIG. 6. Depending on the reference, different numbering schemes are used to define the A1-vWF residues. In this report, the numbering scheme of Cruz et al. (supra) is used that allocates residues 479-717 to the A1-domain (see also FIG. 7). The crystal structure of the complex between the Nanobody 12a2h1 (SEQ ID NO: 19) and the A1 domain of the von Willebrand Factor (A1-vWF) was solved by Proteros (http://www.proteros.com). Recombinantly expressed proteins of A1-vWF and 12a2h1 were supplied by Ablynx and used in a broad crystallization screening.

Crystals were flash-frozen and measured at a temperature of 100K. The X-ray diffraction data of the complex were collected at the SWISS LIGHT SOURCE (SLS, Villigen, Switzerland) using cryogenic conditions. The structure was solved and refined to a final resolution of 1.75 A.

The crystal belongs to space group P 2₁ 2₁ 2₁ and contains 2 essentially identical A1-vWF:12a2h1 complexes (complex A and B) in the asymmetric unit. The resulting electron density shows an unambiguous binding mode for the Nanobody 12a2h1, including the orientation and conformation of the Nanobody.

### Complex Structure

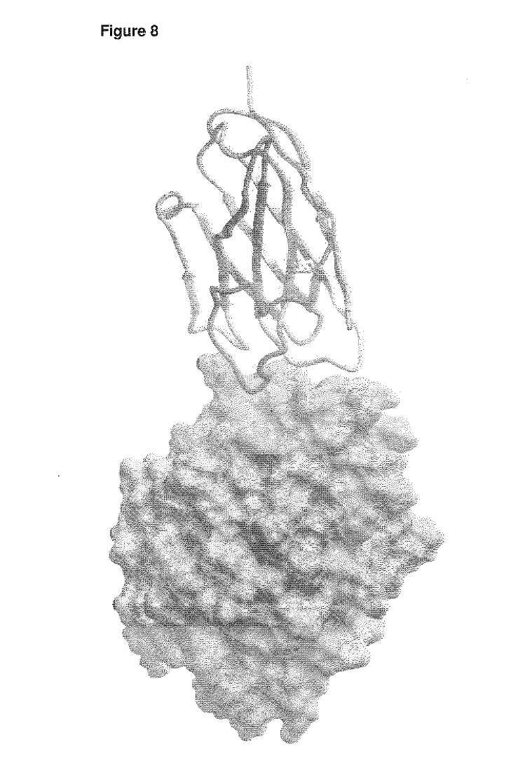

The structure of the A1-vWF:12a2h1 complex is shown in FIG. 8, wherein the surface of the A1-vWF domain is shown in orange and Nanobody 12a2h1 is shown in a ribbon representation with CDR1 in green, CDR2 in cyan and CDR3 in blue. CDR-loops 1, 2 and 3 of the Nanobody tightly interact with A1-vWF and are well defined by the electron density.

For the A1 domain of vWF (amino-acids 479-717) residues Asp-498 to Ala-701 in complex A and residues Ser-500 to Ala-704 in complex B that are covered by the electron density.

Interactions Between 12a2h and A1-vWF

The interaction pattern between the Nanobody 12a2h1 and A1-VWF can be divided in 4 regions: CDR1, CDR2, CDR3 and “CDR4”.

### CDR 1

Five residues of CDR1 show significant interactions with A1-vWF; the main interactions are provided by S30 and Y31:
R27
   1. The side chain of R27 is not well resolved in the X-ray structure and will probably not make crucial interactions with A1-vWF.
   2. The main chain oxygen of R27 forms electrostatic interactions with Arg-545 and Trp-550 of A1-vWF.
T28
   3. Forms Van der Waals interactions with Trp-550 of A1-vWF
F29
   4. The side chain points inwards and is important for internal stability and CDR1 conformation
   5. The main chain oxygen interacts with Arg-545 of A1-vWF
S30
   6. Forms Van der Waals interactions with Tyr-508, Ser-510 and Arg-545 of A1-vWF
Y31
   7. Forms Van der Waals interactions with Ser-500, Pro-502, Pro-503, Tyr-508 and Arg-545 of A1-vW
CDR 2

Three CDR2 residues significantly interact with A1-vWF:
S52
   8. The side chain forms a hydrogen bond with Asp-506 of A1-vWF
R52a
   9. One of the most crucial residues for the interaction between the Nanobody and A1-vWF.
   10. The side chain forms a hydrogen bond with the main chain of Asp-506 of A1-vWF
   11. Interacts with Tyr-508 of A1-vWF
   12. Is also heavily involved in internal hydrogen bonds with CDR1 and CDR3 residues.
T53
   13. Main chain NH and side chain form a hydrogen bond with Asp-506 of A1-vWF
   14. Good Van der Waals interactions with Asp-506 and Pro-503
CDR 3

Four residues of CDR 3 are important:
E100
   15. Shows van der Waals interactions with Phe-507, Tyr-508, Cys-509 and Arg-511 of A1-vWF
   16. The side chain forms a hydrogen bond with the side chain of Arg-511 of A1-vWF.
   17. The main chain oxygen forms a hydrogen bond with the main chain NH of Tyr-508 of A1-vWF
D100a
   18. Forms van der Waals interactions with Phe-507 of A1-vWF.
G100b
   19. The absence of a side chain is important for a good shape complementarity with A1-vWF. It also allows Arg-52a to interact optimally with A1-vWF.
R100c
   20. Van der Waals interactions with Asp-506 of A1-vWF.

### “CDR 4”

The loop region between residues 73 and 76 in framework 3 is also referred to as CDR4. Two residues in this region interact with A1-vWF:
N73
   21. Forms Van der Waals interactions with Pro-505 and Pro-503 of A1-vWF
R76
   22. Interacts with Ser-500 of A1-vWF

The terms and expressions which have been employed are used as terms of description and not of limitation, and there is no intention in the use of such terms and expressions of excluding any equivalents of the features shown and described or portions thereof, it being recognized that various modifications are possible within the scope of the invention.

All of the references described herein are incorporated by reference, in particular for the teaching that is referenced hereinabove.

1. A method for the treatment, a pharmaceutical composition for the treatment of a thromboembolic disorder, or use in the treatment of a thromboembolic disorder in patients in need thereof, wherein said treatment comprises administering
i) an effective dose regimen of an anti vWF agent; and
ii) a low dose regimen of a thrombolytic agent to said patient.

2. A method for the treatment, a pharmaceutical composition for the treatment of a thromboembolic disorder, or use in the treatment of a thromboembolic disorder in patients in need thereof, wherein said treatment comprises administering to said patient an effective dose regimen of an anti vWF agent; and wherein said thromboembolic disorder is characterized by rt-PA resistant thrombi.

3. The method, composition or use of claim 1, wherein the thromboembolic disorder is selected from the group of disorders consisting of myocardial infarction, ischemic stroke, acute ischemic stroke, deep vein thrombosis or pulmonary embolism.

4. The method, composition or use of claim 1, wherein the thromboembolic disorder is acute ischemic stroke.

5. The method, composition or use of claim 1, wherein the anti vWF agent is selected from the group of agents consisting of an A1 vWF binding agent, a polypeptide comprising a single domain antibody with the epitope of 12a2h1, a polypeptide comprising a single domain antibody having a CDR combination of SEQ ID NO: 1, a polypeptide comprising a nanobody having a CDR combination as shown in SEQ ID NO: 1, a polypeptide comprising SEQ ID NO: 1 and a polypeptide having SEQ ID NO: 1.

6. The method, composition or use of claim 1, wherein the anti vWF agent is the polypeptide having SEQ ID NO: 1.

7. The method, composition or use of claim 1, wherein the thrombolytic agent is rt-PA.

8. An amino acid sequence comprising a single domain antibody directed against the epitope of 12a2h1 on vWF, wherein the single domain antibody is not a nanobody or a polypeptide that comprises a nanobody having identical CDRs from any of the nanobodies 12a2 (SEQ ID NO:20), 12a5 (SEQ ID NO:21), or 12b6 (SEQ ID NO:22).

9. The amino acid sequence of claim 8 that consists essentially of the single domain antibody directed against the epitope of 12a2h1 on vWF or a construct thereof.

10. The amino acid sequence of claim 8, wherein the single domain antibody is a nanobody.

11. The method, composition or use of claim 2, wherein the thromboembolic disorder is selected from the group of disorders consisting of myocardial infarction, ischemic stroke, acute ischemic stroke, deep vein thrombosis or pulmonary embolism.

12. The method, composition or use of claim 2, wherein the thromboembolic disorder is acute ischemic stroke.

13. The method, composition or use of claim 2, wherein the anti vWF agent is selected from the group of agents consisting of an A1 vWF binding agent, a polypeptide comprising a single domain antibody with the epitope of 12a2h1, a polypeptide comprising a single domain antibody having a CDR combination of SEQ ID NO: 1, a polypeptide comprising a nanobody having a CDR combination as shown in SEQ ID NO: 1, a polypeptide comprising SEQ ID NO: 1 and a polypeptide having SEQ ID NO: 1.

14. The method, composition or use of claim 2, wherein the anti vWF agent is the polypeptide having SEQ ID NO: 1.

15. The method, composition or use of claim 2, wherein the thrombolytic agent is rt-PA.
